(19) United States
(12) Patent Application Publication
(10) Pub. No.: US 2023/0312630 A1
(43) Pub. Date: Oct. 5, 2023

(54) PHOSPHORAMIDITE SYNTHESIS ON-DEMAND

(71) Applicant: Aarhus Universitet, Aarhus C, Denmark (DK)

(72) Inventors: Alexander Hjortlund SANDAHL, Aarhus V (DK); Thuy Jane Dinh NGUYEN, Brabrand (DK); Kurt Vesterager GOTHELF, Risskov (DK)

(21) Appl. No.: 18/015,282

(22) PCT Filed: Jul. 15, 2021
(86) PCT No.: PCT/EP2021/069851

§ 371 Date: Jan. 9, 2023

(30) Foreign Application Priority Data
Jul. 16, 2020 (EP) 20186197.8

(51) Int. Cl. C07H 1/00; C07F 9/24; C07H 19/16; C07H 19/06; C08F 12/08; C08F 8/40; B01D 15/08

(52) U.S. Cl. CPC C07H 1/00; C07F 9/2408; C07H 19/16; C07H 19/06; C08F 12/08; C08F 8/40; B01D 15/08

(57) ABSTRACT

A process for synthesizing phosphoramidites by immobilizing a phosphitylating agent on an activated resin to create a loaded resin and then bringing the loaded resin into contact with a suitable substrate. The phosphoramidites are synthesized within minutes from applying the starting materials. Thus, the process makes it possible to create specific phosphoramidites on-demand as they are needed in further applications. The substrates to be applied are mostly nucleosides, thus to create nucleoside phosphoramidites for subsequent oligonucleotide synthesis.

### CROSS-REFERENCE TO RELATED APPLICATIONS

This application is a national stage application, filed under 35 U.S.C. 371, of International Patent Application Serial No. PCT/EP2021/069851 Jul. 15, 2021 which claims the benefit of priority to European Patent Application Serial No. 20186197.8, filed Jul. 16, 2020, the entire content of both of which is incorporated herein by reference.

### TECHNICAL FIELD OF THE INVENTION

The present invention relates to chemical synthesis of phosphoramidites. In particular, the present invention relates to synthesis of nucleoside phosphoramidites for subsequent application in oligonucleotide synthesis.

### BACKGROUND OF THE INVENTION

Automated chemical synthesis of oligonucleotides is of fundamental importance in many industries and relies upon the procurement of phosphoramidites. Synthetic oligonucleotides are essential for a range of different areas and millions of oligonucleotides are synthesized daily for use in research laboratories, hospitals and industry. The availability of oligonucleotide sequences through chemical synthesis is one of the corner stones of biotechnology and a prerequisite for technologies such as PCR, DNA-sequencing, synthetic biology and CRISPR-Cas9. Among its many applications PCR is one of the key technologies used for identification of pathogens. By the development of chemical modifications of oligonucleotides the application of antisense oligonucleotide-based strategies for treatment of diseases has become possible. Libraries of vast numbers of oligonucleotide aptamers and microarrays of thousands of oligonucleotides on surfaces are also prepared by chemical synthesis. Furthermore, exploration of new assembly strategies of DNA has laid the groundwork for DNA nanotechnology, where DNA is used as engineering material. Lately, it has also been shown that oligonucleotides can be used as a unit for digital data storage. However, common for all the oligonucleotides described above is that they are synthesized from phosphoramidites.

The crucial phosphoramidite building blocks are still commonly prepared by conventional solution-phase methods using phosphoramidite reagents, such as PNs (e.g. β-cyanoethyl-bis(diisopropylamino)-methoxyphosphine or cyanoethyl bis(diisopropylamino)phosphoramidite). These preparations make use of nucleoside reactants that are protected by an alcohol protecting group, such as dimethoxytrityl (DMTr), and is performed in solution. The reaction times for said preparation methods are typically 1-5 hours and, afterwards purification by column chromatography with triethylamine as part of the eluent is required to prevent hydrolysis of the product. In addition, subsequent removal of the triethylamine is a crucial need, because it otherwise delimits the coupling of the produced phosphoramidites when used in oligonucleotides synthesis. All the steps add up to a total of at least 12 hours of synthesis and purification before the phosphoramidites are ready for oligonucleotide synthesis.

Apart from the many hours of synthesis and purification, phosphoramidites also need to be stored under an inert atmosphere and preferably at −20° C. to minimize oxidation and hydrolysis. However, on oligonucleotide synthesizers, it is only practical to store the phosphoramidites in solution and at ambient temperature. The phosphoramidites are thereby degraded by different autocatalytic reactions and water-catalyzed pathways.

To circumvent the problems related to the stability of phosphoramidites, it has previously been attempted to prepare phosphoramidites in situ. However, the procedures have never gained broad use, since any residues of the PN reagent deteriorate the following synthesis of oligonucleotides.

As such, Nielsen, J. et al., Nucleic Acids Research, 1986, 14, 7391-7403 discloses a preparation of 5 different deoxyribonucleoside phosphoramidites by stirring a mixture of the dissolved reactants for 0.5 to 20 hours. The mixture is left to precipitate a diisopropylammonium tetrazolide salt, which is an integrated side-product of the procedure. The salt is removed by filtration before phosphoramidites, dimers (5%) and hydrolysis products (5-10%) are pumped into the solid phase synthesizer for creating oligonucleotides.

Thus, all steps of synthesis, purification and storage are somehow troubled by the ease at which phosphoramidites undergo autocatalytic and water-catalyzed hydrolytic reactions. The present methods for providing phosphoramidites are therefore highly cost intensive, detrimental to new developments within the biotechnological fields, and decreases the efficiency at which PCR can be used to identify pathogens, such as bacteria and virus.

A quick, on-demand synthesis of phosphoramidites from their corresponding alcohols in quantitative yields and without the need of purification before being submitted directly to automated oligonucleotides (ON) synthesis, would preferably remove all of the problems listed above.

Hence, an improved method for synthesis of phosphoramidites would be advantageous, and in particular a much faster synthesis of phosphoramidites would be advantageous.

### SUMMARY OF THE INVENTION

Thus, an object of the present invention relates to an on-demand process for providing phosphoramidites of high purity and within minutes. The process is based on immobilizing a phosphitylating agent on an activated resin, thus to create a loaded resin with phosphitylating properties. A substrate is then brought into contact with the loaded resin, whereby the desired phosphoramidites take form. The synthesis is completed once the phosphoramidites on the loaded resin are eluted and collected.

In particular, it is an object of the present invention to provide a very fast process for the synthesis of pure phosphoramidites, and which process solves the above mentioned problems of the prior art wherein unwanted reactions lead to impurities, low yields, and requires subsequent purification steps. The process of the present invention, wherein the phosphitylating agent is immobilized on an activated resin to form a loaded resin, makes it possible to remove salts and excess reagents from the loaded resin before bringing it into contact with the substrate. The following reaction between the loaded resin and the substrate is so fast that it essentially outcompetes any unwanted formation of impurities, e.g. by hydrolysis, oxidation, polymerization, or degradation reactions. Thus, the desired phosphoramidites are collected within minutes from applying the substrate to the loaded resin. The high purity of the produced phosphoramidites allows for their immediate introduction into other areas of application, such as automated ON synthesis.

A first aspect of the present invention relates to a process for providing phosphoramidites, which process comprises the steps of:
a) providing an activated resin of a resin, comprising one or more of the heterocyclic moieties selected from the group consisting of imidazole, pyrazole, 1,2,3-triazole, 1,2,4-triazole, tetrazole, 2-nitrophenyltetrazole, 4-nitrophenyltetrazole, 2,4-dinitrophenyltetrazole, 3-nitro-1,2,4-triazole, 4,5-dicyanoimidazole, and 4-nitroimidazole,
b) obtaining a loaded resin by contacting the activated resin with a first solution comprising a first reactant according to Formula (1)

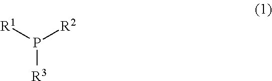

wherein, R¹ and R² are independently selected from the group consisting of di(C₁-C₆)alkylamino, pyrrolidino, morpholino, bromo, iodo, and chloro; R³ is selected from the group consisting of (C₁-C₆)alkyl, (C₁-C₆)alkoxy, 2-cyanoethoxy, benzoylthioethylthio, and 1,1-dimethyl-2-cyanoethoxycarbonylmethyl, or the first reactant is P-chloro-1-methyl-1-phenyl-2-oxa-3-phospha-tetrahydropyrrolizine,
c) contacting a second solution comprising a second reactant with the loaded resin of step b),
d) collecting the phosphoramidites,

wherein, the second reactant is selected from the group consisting of nucleosides, nucleoside analogs, synthetic nucleosides, disulfides, derivatives of glycols, azides, and ethylene glycol oligomers.

Another aspect of the present invention relates to an activated resin of a resin, comprising one or more of the heterocyclic moieties selected from the group consisting of imidazole, pyrazole, 1,2,3-triazole, 1,2,4-triazole, tetrazole, 2-nitrophenyltetrazole, 4-nitrophenyltetrazole, 2,4-dinitrophenyltetrazole, 3-nitro-1,2,4-triazole, 4,5-dicyanoimidazole, and 4-nitroimidazole.

Yet another aspect of the present invention relates to a loaded resin comprising a resin connected to one or more of the heterocyclic moieties selected from the group consisting of imidazole, pyrazole, 1,2,3-triazole, 1,2,4-triazole, tetrazole, 2-nitrophenyltetrazole, 4-nitrophenyltetrazole, 2,4-dinitrophenyltetrazole, 3-nitro-1,2,4-triazole, 4,5-dicyanoimidazole, and 4-nitroimidazole, wherein each heterocyclic moiety is further connected to a phosphoramidite moiety.

Still another aspect of the present invention relates to a loaded resin obtained by a process comprising the steps of:
a) providing an activated resin of a resin, comprising one or more of the heterocyclic moieties selected from the group consisting of imidazole, pyrazole, 1,2,3-triazole, 1,2,4-triazole, tetrazole, 2-nitrophenyltetrazole, 4-nitrophenyltetrazole, 2,4-dinitrophenyltetrazole, 3-nitro-1,2,4-triazole, 4,5-dicyanoimidazole, and 4-nitroimidazole,
b) obtaining the loaded resin by contacting the activated resin with a first solution comprising a first reactant according to Formula (7)

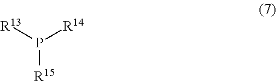

wherein, R¹³ and R¹⁴ are independently selected from the group consisting of di(C₁-C₆)alkylamino, pyrrolidino, morpholino, bromo, iodo, and chloro; R¹⁵ is selected from the group consisting of (C₁-C₆)alkyl, (C₁-C₆)alkoxy, 2-cyanoethoxy, benzoylthioethylthio, and 1,1-dimethyl-2-cyanoethoxycarbonylmethyl, or the first reactant is P-chloro-1-methyl-1-phenyl-2-oxa-3-phospha-tetrahydropyrrolizine.

### BRIEF DESCRIPTION OF THE FIGURES

FIG. 1 shows, the general concept of the on-demand synthesis, wherein an alcohol substrate reacts with a loaded resin to produce the corresponding phosphoramidite. The loaded resin is returned to its activated resin stage, which may be reloaded upon contact with a phosphitylating agent (PCI).

FIG. 2 shows, a schematic representation of the test system setup, wherein a pump is flowing a solvent, such as dichloromethane (DCM), through a tubing system comprising three loops for injection of methanol (MeOH), phosphitylating agent (PCI) and alcohol substrate, respectively. The tubing system is terminated by a compartment comprising resin, after which, the phosphoramidite products are collected.

FIGS. 3 to 14 show, structures of the produced phosphoramidites in insert A). Insert B) shows, ¹H NMR spectra of characteristic peaks of the products and the starting materials in the collected samples for each separate experiment, and for the pure starting materials, and for the pure reference products. Decreasing flow rates were used for the separate experiments leading to increased residence times (1, 2, 4, 6, or 8 minutes) for the reactants in the resin compartment.

FIG. 3 relates to produced phosphoramidite, T. It can be seen that by using a flow rate leading to 8 minutes in the resin compartment yields the phosphoramidite, T, in near-quantitative yield.

FIG. 4 relates to produced phosphoramidite, C. It can be seen that by using a flow rate leading to 6 minutes in the resin compartment yields the phosphoramidite, C, in near-quantitative yield.

FIG. 5 relates to produced phosphoramidite, A. It can be seen that by using a flow rate leading to 8 minutes in the resin compartment yields the phosphoramidite, A, in near-quantitative yield.

FIG. 6 relates to produced phosphoramidite, G. It can be seen that by using a flow rate leading to 1 minute in the resin compartment yields the phosphoramidite, G, in near-quantitative yield.

FIG. 7 relates to produced phosphoramidite, 1. It can be seen that by using a flow rate leading to 2 minutes in the resin compartment yields the phosphoramidite, 1, in near-quantitative yield.

FIG. 8 relates to produced phosphoramidite, 2. It can be seen that by using a flow rate leading to 4 minutes in the resin compartment yields the phosphoramidite, 2, in near-quantitative yield.

FIG. 9 relates to produced phosphoramidite, 3. It can be seen that by using a flow rate leading to 4 minutes in the resin compartment yields the phosphoramidite, 3, in near-quantitative yield.

FIG. 10 relates to produced phosphoramidite, 4. It can be seen that by using a flow rate leading to 6 minutes in the resin compartment yields the phosphoramidite, 4, in near-quantitative yield.

FIG. 11 relates to produced phosphoramidite, 5. It can be seen that by using a flow rate leading to 4 minutes in the resin compartment yields the phosphoramidite, 5, in near-quantitative yield. FIG. 11 also comprise an insert C) showing ¹⁹F NMR spectra of characteristic peaks.

FIG. 12 relates to produced phosphoramidite, 6. It can be seen that by using a flow rate leading to 2 minutes in the resin compartment yields the phosphoramidite, 6, in near-quantitative yield.

FIG. 13 relates to produced phosphoramidite, 7. It can be seen that by using a flow rate leading to 1 minute in the resin compartment yields the phosphoramidite, 7, in near-quantitative yield.

FIG. 14 relates to produced phosphoramidite, 8.1. It can be seen that by using a flow rate leading to 1 minutes in the resin compartment yields the phosphoramidite, 8.1, in near-quantitative yield.

FIG. 15 shows, a comparison between a 13-mer oligonucleotide (ON) prepared by automated ON synthesis using phosphoramidites obtained from the on-demand synthesis or from a conventional method. The inserted HPLC chromatograms illustrate that an oligonucleotide of sequence 5′-TACGTGACCTGAT-3′ can be prepared from the on-demand synthesized phosphoramidites as a product, which, is at least as pure as the same product produced by the conventional method.

FIG. 16 shows, steps in the procedure to create a modified NMR tube.

FIGS. 17 to 21 show, in inserts A) structures of molecules engineered to match the exposed part of a loaded resin, and in inserts B) structures of the corresponding loaded resins. Inserts C) show, two stacked ³¹P NMR spectra for characteristic peaks. The lower spectrum is of the structures depicted in A), whereas the upper spectrum is of the loaded resins in B). The characteristic NMR peaks for the P atom shifts or splits when connected to a specific resin.

FIG. 17 shows, the characteristic ³¹P NMR peaks for a loaded resin (gel phase) comprising imidazole.

FIG. 18 shows, the characteristic ³¹P NMR peak for a loaded resin (gel phase) comprising 1,2,3-triazole.

FIG. 19 shows, the characteristic ³¹P NMR peaks for a loaded resin (gel phase) comprising tetrazole.

FIG. 20 shows, the characteristic ³¹P NMR peaks for a loaded resin (gel phase) comprising 1,2,4-triazole.

FIG. 21 shows, the characteristic ³¹P NMR peaks for a loaded resin (gel phase) comprising 3-nitro-1,2,4-triazole.

FIG. 22 shows, structures of the produced phosphoramidites when using the PCI, chloro(diisopropylamino)(methyl)phosphine. Insert A) shows the product obtained when the substrate is T-alc, insert B) when using A-alc, C) when using G-alc.

FIGS. 23 to 24 show, in inserts A) structures of molecules engineered to match the exposed part of a loaded resin, and in inserts B) structures of the corresponding loaded resins. Inserts C) show, two stacked ³¹P NMR spectra for characteristic peaks. The lower spectrum is of the structures depicted in A), whereas the upper spectrum is of the loaded resins in B). The characteristic NMR peaks (chemical shifts) for the P atom shifts when connected to a specific resin.

FIG. 23 shows, characteristic ³¹P NMR peak changes of a chloro(diisopropylamino)(1,1-dimethyl-2-cyanoethoxycarbonylmethyl)phosphine loaded resin (gel phase) when compared to a solution phase reference.

FIG. 24 shows, characteristic ³¹P NMR peak changes of a chloro(diisopropylamino)(methyl)phosphine loaded resin (gel phase) when compared to a solution phase reference.

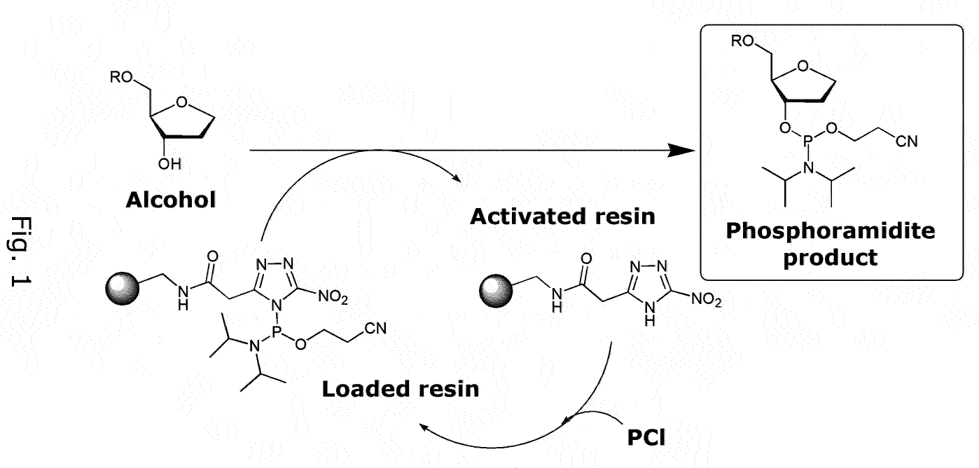

The present invention will now be described in more detail in the following.

### DETAILED DESCRIPTION OF THE INVENTION

### Definitions

Prior to discussing the present invention in further details, the following terms and conventions will first be defined:

Azide

In the present context, the term “azide” refers to an organic compound comprising at least one azide group (i.e. an —N₃ group).

Alkylidene Bridge

In the present context, the term “alkylidene bridge” refers to an alkyl chain, which is forming a bridge between two atoms in a bicyclic molecule.

Disulfide

In the present context, the term “disulfide” refers to a compound comprising at least one moiety composed of two sulfur atoms that are directly bonded two each other (i.e. a moiety of —S—S—).

Ethylene Glycol Oligomer

In the present context, the term “ethylene glycol oligomer” refers to a polymer composed of a finite number of —OCH₂CH₂— repeating units, wherein the number of repeating units is a number between 2 and 100.

Nitrogen Heterocycle

In the present context, the term “nitrogen heterocycle” refers to a compound comprising at least one ring of atoms and, wherein at least one ring of atoms comprises at least one nitrogen atom.

Nucleoside

In the present context, the term “nucleoside” refers to an optionally substituted compound comprising a 5-membered ring of a five-carbon sugar (e.g. ribose, deoxyribose), and which ring is bonded to a nitrogen heterocycle, such as a nucleobase. A nucleoside possesses the ability to form base pairs with another nucleoside as in e.g. DNA or RNA.

Nucleotide

In the present context, the term “nucleotide” refers to a nucleoside, nucleoside analog, or synthetic nucleoside, which is attached to a phosphate group.

Nucleoside Analog

In the present context, the term “nucleoside analog” refers to a structure similar to a nucleoside, but wherein the 5-membered ring has been modified. For example, the modification may be the exchange of an oxygen atom in said ring with an atom of C, N, or S, or an exchange of one or more of substituents on the ring with other substituents.

Oligonucleotide

In the present context, the term “oligonucleotide” refers to an oligomer composed of a sequence of nucleotide residues. The number of nucleotide residues in an oligonucleotide may be between 2 and 200.

Optionally Substituted

In the present context, the term “optionally substituted” refers to a chemical structure wherein one or more of the hydrogen atoms may, optionally, be exchanged with substituents (e.g. hydroxy, oxo, etc.).

Phosphoramidites

In the present context, the term “phosphoramidites” refers to compounds comprising a moiety wherein a phosphorous(III) atom is bonded to two oxygen atoms and one nitrogen atom. However, the term also includes thiophosphoramidites wherein one of the two oxygen atoms in the moiety is replaced with a sulfur atom.

Protecting Group

In the present context, the term “protecting group” refers to a group or moiety in a compound, which is stable towards specific reagents and/or chemical conditions. A part of a molecule may be replaced or substituted with a protecting group by chemical modification using a protecting agent. Later, the replacement or substitution may be removed to reform the original part of the molecule by treatment with a reagent suitable for the purpose or by changing to specific reaction conditions. For example, treatment of a hydroxy group with an alcohol protecting agent substitutes the hydrogen of the hydroxy, or the entire hydroxy group, with an alcohol protecting group.

Radical

In the present context, a radical is a chemical moiety obtained by removing a H from the chemical structure of a compound whereby a covalent bond is broken and a first electron and a second electron (the electrons originally forming the bond) are divided such that the first electron is removed together with the H, whereas the second electron stays with the newly formed radical. The radical may subsequently form a new covalent bond at the location within the chemical structure where the H was removed, thus connecting the radical with another chemical group, molecule, moiety, unit, compound, radical, diradical, species, substance, or similar.

Resin

In the present context, the term “resin” refers to a solid or highly viscous material optionally comprising pores and void spaces. A resin may be an organic polymer or an inorganic material. In addition, the term “activated resin” refers to a resin which has been chemically modified to comprise specific heterocyclic moieties. In addition, the term “loaded resin” refers to an activated resin which has been further modified to comprise phosphitylating moieties attached to said heterocyclic moieties.

Synthetic Nucleoside

In the present context, the term “synthetic nucleoside” refers to a compound similar to a nucleoside, but wherein the 5-carbon ring is replaced with a cyclic or acyclic moiety derived from a 4, 5, or 6 carbon sugar, amino acid or amino acid derivative.

The number of carbon atoms of a chemical structure or a moiety thereof, may be announced in a parenthesis as a Cₓ-C_{y} range inserted prior to the structure or moiety to which it refers. For example, (C₁-C₆)alkoxy refers to linear and branched alkoxy groups comprising a number of carbon atoms selected from that range (examples are: methoxy, ethoxy, and isopropoxy). In another example, di(C₁-C₅)alkylamino refers to an amino functional group carrying two alkyl groups and which alkyl groups comprise a number of carbon atoms independently selected from the range shown in the parenthesis (examples are: dimethylamino, diisopropylamino, and methylpentylamino).

The process of the present invention makes use of a resin, which has been modified to possess a specific chemical activity against compounds comprising phosphorous(III). The modification is a change of the reactive sites on the exposed and terminal positions within the resin to reactive sites possessing one or more heterocyclic moieties selected from a group of 5-membered nitrogen heterocyclic moieties, which were found to exercise the required chemical activity. The initial part of the process of the present invention is therefore to provide an activated resin which is then reacted with a first reactant comprising phosphorous(III). The first reactant is a phosphitylating agent and upon reaction the properties of the agent is transferred to the activated resin whereby a loaded resin with phosphitylating properties is obtained. The loaded resin is considered a very important feature of the present invention and key to success for the on-demand synthesis of phosphoramidites. The loaded resin, with the immobilized phosphitylating agent, may be washed with solvent before it is brought into contact with a second reactant and thus, the reaction between the substrate and the loaded resin may be a very clean reaction producing phosphoramidites of high purity after only few minutes of contact.

A first aspect of the present invention relates to a process for providing phosphoramidites, which process comprises the steps of:
a) providing an activated resin of a resin, comprising one or more of the heterocyclic moieties selected from the group consisting of imidazole, pyrazole, 1,2,3-triazole, 1,2,4-triazole, tetrazole, 2-nitrophenyltetrazole, 4-nitrophenyltetrazole, 2,4-dinitrophenyltetrazole, 3-nitro-1,2,4-triazole, 4,5-dicyanoimidazole, and 4-nitroimidazole,
b) obtaining a loaded resin by contacting the activated resin with a first solution comprising a first reactant according to Formula (1)

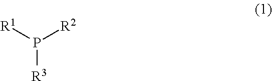

wherein, R¹ and R² are independently selected from the group consisting of di(C₁-C₆)alkylamino, pyrrolidino, morpholino, bromo, iodo, and chloro; R³ is selected from the group consisting of (C₁-C₆)alkyl, (C₁-C₆)alkoxy, 2-cyanoethoxy, benzoylthioethylthio, and 1,1-dimethyl-2-cyanoethoxycarbonylmethyl, or the first reactant is P-chloro-1-methyl-1-phenyl-2-oxa-3-phospha-tetrahydropyrrolizine,
c) contacting a second solution comprising a second reactant with the loaded resin of step b),
d) collecting the phosphoramidites,

wherein, the second reactant is selected from the group consisting of nucleosides, nucleoside analogs, synthetic nucleosides, disulfides, derivatives of glycols, azides, and ethylene glycol oligomers.

The inventors of the present invention were surprised to find that by immobilizing a phosphitylating agent on an activated resin and subsequently bringing it into contact with the second reactant, they were able to create the corresponding phosphoramidites in a very high yield within only 15 minutes of contact.

Thus, an embodiment of the present invention relates to the process, wherein the second solution and the loaded resin in step c) are in contact with each other for 0.1 to 15 minutes. A more specific embodiment of the present invention relates to the process, wherein the second solution and the loaded resin in step c) are in contact with each other for 0.1 to 13 minutes, such as 1 to 13 minutes, such as 2 to 13 minutes, 3 to 13 minutes, such as 4 to 13 minutes, such as 5 to 13 minutes, such as for 0.1 to 10 minutes, preferably for 0.1 to 8 minutes.

The process of the present invention can be setup as a flow process coupled to automated oligonucleotide synthesis, whereby specific oligonucleotides may be synthesized on-demand. Thus, a quick and efficient method to prepare phosphoramidites is considered important for society, not just in regard of the produced phosphoramidites, but in particular also in regard of the on-demand provision of oligonucleotides which are corner stones within many disciplines of biotechnology and a prerequisite for technologies such as PCR, DNA-sequencing, synthetic biology and CRISPR-Cas9.

Therefore, an embodiment of the present invention relates to the process, wherein the contacting of the second solution with the loaded resin in step c) is done by flowing the second solution through the loaded resin. A further embodiment of the present invention relates to the process, wherein steps b) and/or c) are conducted using a column.

As mentioned, the process of the present invention may be integrated into DNA synthesizers whereby the conventional manual synthesis and storage of phosphoramidites are avoided.

An embodiment of the present invention therefore relates to the process, wherein the phosphoramidites are at a purity which allows for direct utilization of the phosphoramidites in synthesis of oligonucleotides.

A particular embodiment of the present invention relates to the process, wherein the phosphoramidites are at a purity measured with HPLC of at least 90.0%, such as at least 95.0%, such as at least 98.0%, such as at least 99.0%, preferably such as at least 99.5%.

The phosphitylating agent (PCI) is a compound comprising a phosphorous atom in oxidation state III which is surrounded by three ligands.

An embodiment of the present invention relates to the process, wherein the first reactant is selected from the group consisting of
chloro(diisopropylamino)(2-cyanoethoxy)phosphine,
chloro(pyrrolidino)(benzoylthioethylthio)phosphine,
bis(diisopropylamino)(2-cyanoethoxy)phosphine,
chloro(diisopropylamino)(1,1-dimethyl-2-cyanoethoxycarbonylmethyl)phosphine,
bis(diisopropylamino)(1,1-dimethyl-2-cyanoethoxycarbonylmethyl)phosphine,
chloro(diisopropylamino)(methyl)phosphine,
bis(diisopropylamino)(methyl)phosphine, and
P-chloro-1-methyl-1-phenyl-2-oxa-3-phospha-tetrahydropyrrolizine.

The relationships between name and structure for some of the phosphitylating agents are:

| | |
|---|---|
| chloro(diisopropylamino)(2-cyanoethoxy)phosphine | 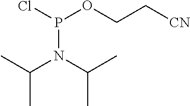 |
| bis(diisopropylamino)(2-cyanoethoxy)phosphine | 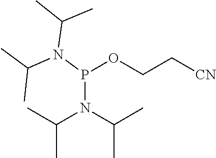 [SMILES: CC(C)N(C(C)C)P(OCCC#N)N(C(C)C)C(C)C] |
| chloro(diisopropylamino)(1,1-dimethyl-2- cyanoethoxycarbonylmethyl)phosphine | 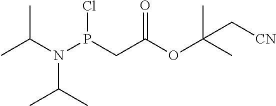 [SMILES: CC(C)N(C(C)C)P(Cl)CC(=O)OC(C)(C)CC#N] |
| chloro(diisopropylamino)(methyl)phosphine | 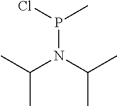 |
| P-chloro-1-methyl-1-phenyl-2-oxa-3-phospha- tetrahydropyrrolizine* | 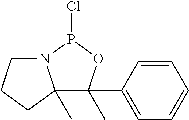 [SMILES: CC1(c2ccccc2)OP(Cl)N2CCCC21C] |
| (1R,7aS)-P-chloro-1-methyl-1-phenyl-2-oxa-3- phospha-tetrahydropyrrolizine | 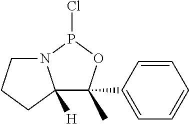 [SMILES: C[C@]1(c2ccccc2)OP(Cl)N2CCC[C@H]21] |
| chloro(pyrrolidino)(benzoylthioethylthio)phosphine | 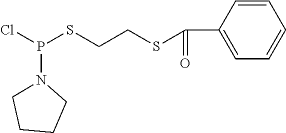 [SMILES: O=C(SCCSP(Cl)N1CCCC1)c1ccccc1] |
| bis(diisopropylamino)(methyl)phosphine | 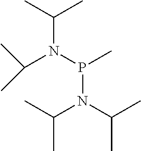 [SMILES: CC(C)N(C(C)C)P(C)N(C(C)C)C(C)C] |
| bis(diisopropylamino)(1,1-dimethyl-2- cyanoethoxycarbonylmethyl)phosphine | 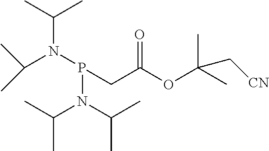 [SMILES: CC(C)N(C(C)C)P(CC(=O)OC(C)(C)CC#N)N(C(C)C)C(C)C] |

*including all isoforms thereof.

A preferred embodiment of the present invention relates to the process, wherein the first reactant is chloro(diisopropylamino)(2-cyanoethoxy)phosphine:

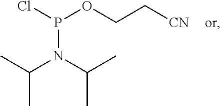

bis(diisopropylamino)(2-cyanoethoxy)phosphine:

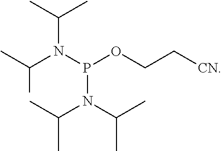 [SMILES: CC(C)N(C(C)C)P(OCCC#N)N(C(C)C)C(C)C]

The activated resin, which is to react with the phosphitylating agent to create the loaded resin having phosphitylating properties, may be based on any resin having functional groups that can react with another functional group in a compound comprising a heterocyclic moiety. An embodiment of the present invention relates to the process, wherein the resin is selected from the group consisting of polystyrene (PS), (aminomethyl)polystyrene (AM-PS), polyethylene glycol (PEG), silica, polyacrylamide with PEG branching, PS with PEG branching, and mixtures thereof. In one embodiment of the present invention relating to the process as described herein, the resin is chosen from the group of resins having amino functional groups. Thus, a particular embodiment of the present invention relates to the process as described herein, wherein the resin is selected from the group consisting of amino functionalized alkyl grafted polystyrene (such as (aminomethyl)polystyrene (AM-PS), and such as (aminobutyl)polystyrene), amino functionalized PEG grafted polystyrene (such as TentaGel™ S—NH₂, and such as HypoGel® NH₂ Resin), amino functionalized PEG grafted modified polystyrene (such as TentaGel® XV HMPA), amino functionalized silica (such as Amino-Synbase™controlled pore glass), amino functionalized PEG such as Aminomethyl ChemMatrix® (AM-CM), and amino functionalized PEG branched polyacrylamide.

The amino functional groups are able to react with compounds comprising functional groups suitable for that purpose, such as a carbonyl group. One embodiment of the present invention relates to the process, wherein the activated resin is provided by reacting a resin having amino functional groups with a compound comprising a carbonyl functional group and a heterocyclic moiety selected from the group consisting of imidazole, pyrazole, 1,2,3-triazole, 1,2,4-triazole, tetrazole, 2-nitrophenyltetrazole, 4-nitrophenyltetrazole, 2,4-dinitrophenyltetrazole, 3-nitro-1,2,4-triazole, 4,5-dicyanoimidazole, and 4-nitroimidazole. In addition, the group of heterocyclic moieties includes any tautomers that may exist thereof.

A particular embodiment of the present invention relates to the process, wherein the activated resin is selected from the group consisting of the following activated resins:

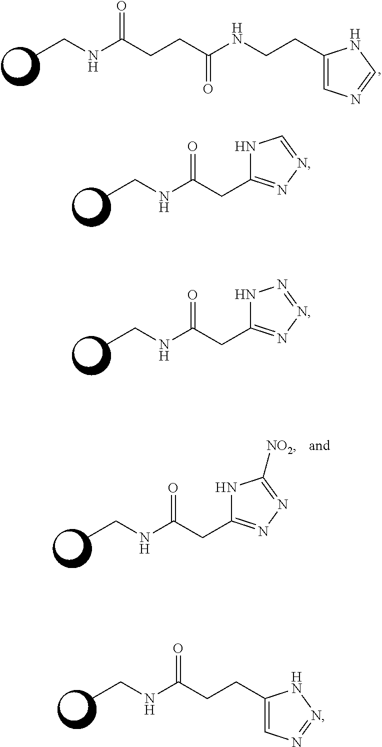 [SMILES: CCNC(=O)CCC(=O)NCCc1cnc[nH]1 ; CCNC(=O)Cc1nnc[nH]1 ; CCNC(=O)Cc1nnn[nH]1 ; CCNC(=O)Cc1nnc([N+](=O)[O-])[nH]1 ; CCNC(=O)CCc1cnn[nH]1]

wherein ( [symbol] ) is the resin.

A preferred embodiment of the present invention relates to the process, wherein the activated resin comprises tetrazole or 3-nitro-1,2,4-triazole moieties.

Second Reactant:

The second reactant is a molecule comprising at least one functional group, which is able to react with the loaded resin to create the corresponding phosphoramidites. For most embodiments, the functional group on the second reactant is a hydroxy group, whereby the second reactant is referred to as a substrate alcohol. Preferably, the second reactant does not comprise more than one hydroxy, because other hydroxy groups could possibly also react with the loaded resin and thereby deteriorate synthesis of the target product. Thus, an embodiment of the present invention relates to the process, wherein the second reactant comprises one —OH group.

A second reactant according to the present invention may not initially fulfill the criteria for being a preferred second reactant. Thus, an embodiment of the present invention relates to the process, wherein if the second reactant comprises more than one of —OH groups, then before the contacting of step c, the more than one of these groups are modified by substitution of the hydrogen atom of each —OH with an alcohol protecting group.

The alcohol protecting groups are chemical moieties or groups that do not react under the chemical conditions during synthesis of the phosphoramidites. However, after a product has been collected an alcohol protecting group may be changed back into a hydroxy group by specific chemical modifications. An embodiment of the present invention relates to the process, wherein the alcohol protecting groups are independently selected from the group consisting of 4,4′-dimethoxytrityl (DMTr), 4-methoxytrityl (MMTr), trityl (Tr), t-butyldimethylsilyl (TBDMS), t-butylsilyl (TBS), bis(2-acetoxyethoxy)methyl (ACE), 1,1-dioxo-thiomorpholin-4-thiocarbonyl, methoxymethyl (MOM), and tri-iso-propylsilyloxymethyl (TOM). A hydroxy group can be modified into an alcohol protecting group by reacting it with an alcohol protecting agent selected from, but not limited to, the group consisting of 4,4′-dimethoxytrityl chloride (DMTr-Cl), 4-methoxytriphenylmethyl chloride (MMTr-Cl), tritylchloride (Tr-Cl), tert-butyldimethylsilyl chloride (TBDMS-Cl), Chloromethyl methyl ether (MOM-Cl), tri-iso-propylsilyloxymethyl (TOM-Cl).

The alcohol protecting group, 4,4′-dimethoxytrityl (DMTr), is widely used herein and is a radical according to the chemical formula:

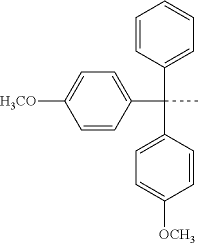

wherein the dotted line denotes the point of attachment where DMTr binds to an oxygen atom in the compound that is protected.

Among the preferred second reactants are those that do not comprise any primary amino groups. This is also to avoid any deteriorating side reactions caused by multiple reaction sites. Thus, an embodiment of the present invention relates to the process, wherein if the second reactant comprises —NH₂ or —NH— groups, then before the contacting of step c, these groups may be modified by substitution of one or both of the hydrogen atoms of each of the —NH₂ or —NH— groups with an amine protecting group.

The amine protecting groups are chemical moieties or groups that do not react under the chemical conditions during synthesis of the phosphoramidites. However, after a product has been collected an amine protecting group may be changed back into the corresponding amine by specific chemical modifications. An embodiment of the present invention relates to the process, wherein the amine protecting groups are independently selected from the group consisting of acetyl (Ac), isobutyryl (iBu), dimethylaminomethylene (dmf), phenoxyacetyl, p-isopropyl-phenoxyacetyl, p-tert-butyl-phenoxyacetyl, trifluoroacetyl, and benzoyl (Bz). An amino group can be modified into an amine protecting group by reacting it with an amine protecting agent selected from, but not limited to, the group consisting of acetic anhydride, isobutyryl chloride, isobutyric anhydride, N,N-dimethylformamide dimethyl acetal, phenoxyacetyl chloride, p-isopropyl-phenoxyacetyl chloride, p-tert-butyl-phenoxyacetyl chloride, trifluoroacetic anhydride, benzoyl chloride, and benzoic anhydride.

A particular embodiment of the present invention relates to the process, wherein the second reactant is a compound according to a Formula selected from the group consisting of Formula (2), Formula (3), Formula (4), Formula (5), and Formula (6),

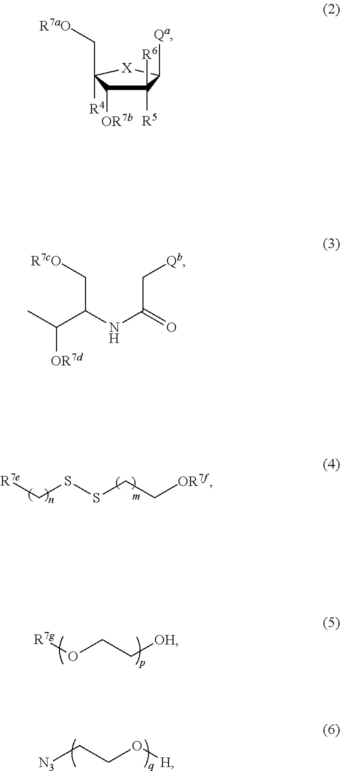

wherein, X is selected from the group consisting of —O—, —S—, —CH₂— and —NH—;
Q^{a} and Q^{b} are independently selected from the group consisting of hydrogen, and optionally substituted nitrogen heterocycle;
R⁴ is hydrogen or an optionally substituted (C₁-C₂)alkylidene bridge forming a ring together with R⁵; R⁵ is selected from the group consisting of —H, —OR⁸, —CH₃, —OCH₃, —OCH₂CH₃, —OCH₂CH₂OCH₃, —F, —Cl, —Br, and —I or forming a ring together with R⁴;
R⁶ is selected from the group consisting of —H, —OR⁹, —CH₃, —OCH₃, —F, —Cl, —Br, and —I; and
if R^{7a} is hydrogen then R^{7b} is an alcohol protecting group,
if R^{7b} is hydrogen then R^{7a} is an alcohol protecting group;
if R^{7c} is hydrogen then R^{7d} is an alcohol protecting group,
if R^{7d} is hydrogen then R^{7c} is an alcohol protecting group;
if R^{7e} is hydrogen then R^{7f} is an alcohol protecting group,
if R^{7f} is hydrogen then R^{7e} is an alcohol protecting group;
R^{7g}, R⁸ and R⁹ are alcohol protecting groups;
p and q are integers independently selected from the group consisting of 2, 3, 4, 5, 6, 7, 8 and 9; n and m are integers independently selected from the group consisting of 1, 2, 3, 4, 5, 6, 7, and 8.

An embodiment of the present invention relates the process, wherein X is selected from the group consisting of —O—, —S—, —CH₂— and —NR¹⁶—, R¹⁶ is an amine protecting group.

A further embodiment of the present invention relates to the process, wherein the nitrogen heterocycle is a radical of a compound selected from the group consisting of adenine, cytosine, guanine, thioguanine, thymine, uracil, xanthine, purine, pyrimidine, pyridazine, pyridine, pyrazine, triazine, pyrrole, pyrazole, imidazole, triazole, pyrrolopyrimidine, pyrazole[1,5-a]pyrimidine, azaindole, benzimidazole, phenoxazine, thiophenoxazine, indazole, indole, indoline, pyrrolopyrrole, quinoline, isoquinoline, theobromine, caffeine, uric acid, isoguanine, isocytosine, and hypoxanthine.

An embodiment of the present invention relates to the process, wherein the nitrogen heterocycle is preferably a radical of a nitrogenous base such as adenine, such as cytosine, such as guanine, such as thymine, and such as uracil.

An embodiment of the present invention relates to the process, wherein the nitrogen heterocycle comprises one or more optional substituents independently selected from the group consisting of hydrogen, (C₁-C₆)alkyl, (C₂-C₆)alkenyl, (C₂-C₆)alkynyl, (C₁-C₆)alkoxy, (C₁-C₆)alkylcarbonylamino, di(C₁-C₅)alkylaminomethaniminyl, phenoxyacetylamino, phenoxyacetylamino, p-isopropyl-phenoxyacetylamino, p-tert-butyl-phenoxyacetylamino, benzoylamino, 4,4′-dimethoxytrityloxy, t-butyldimethylsilyloxy, t-butylsilyloxy, bis(2-acetoxyethoxy)methoxy, 1,1-dioxo-thiomorpholin-4-thiocarbonyloxy, tri-iso-propylsilyloxymethoxy, trifluoromethyl, phenylcarbonyl, phenylcarbonylamino, isopropylcarbonyl, oxo, nitro, cyano, fluoro, chloro, bromo, and iodo. An embodiment of the present invention relates to the process, wherein the optional substituents on the alkylidene bridge is selected from the group consisting of hydrogen fluoro, chloro, bromo, and iodo.

A particularly preferred embodiment of the present invention relates to the process, wherein the second reactant is selected from the group consisting of:

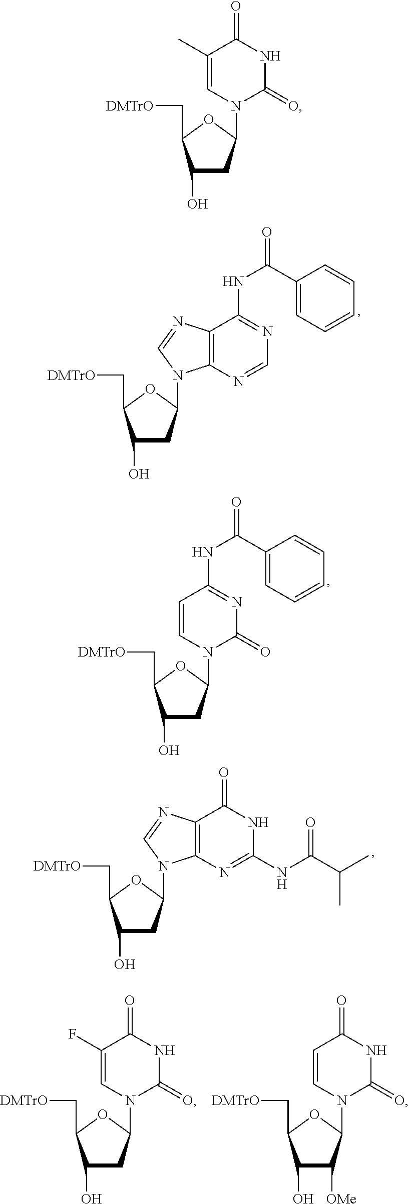

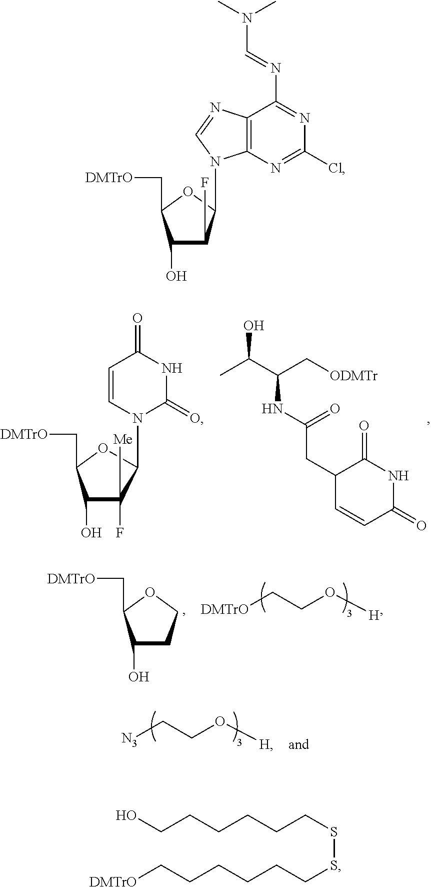

wherein DMTr is 4,4′-dimethoxytrityl.

Reaction of Step b:

An embodiment of the present invention relates to the process, wherein the first solution in step b) comprises a base.

An embodiment of the present invention relates to the process, wherein the first solution in step b) comprises a base selected from the group consisting of triethylamine, N-methylmorpholine, 4-(dimethylamino)pyridine, and N,N-diisopropylethylamine (DIPEA).

An embodiment of the present invention relates to the process, wherein the first solution in step b) comprises a base at a concentration in the range of 0.001 M-0.5 M, such as 0.001M-0.3 M, such as 0.01 M-0.2 M, such as 0.01 M-0.2 M, preferably such as 0.05 M-0.15 M.

An embodiment of the present invention relates to the process, wherein the first solution in step b) comprises a solvent selected from the group consisting of dichloromethane, tetrahydrofuran, diethylether, ethyl acetate, toluene, N,N-dimethylformamide, N-methyl-2-pyrrolidinone, acetonitrile, hexane, and heptane.

The loaded resin may be washed before further use to ensure complete removal of excess amounts of the first solution and removal of any impurities that may have occurred during loading of the activated resin. The possibility of washing the loaded resin is considered important for obtaining a clean product after the following steps. Thus, an embodiment of the present invention relates to the process, wherein the loaded resin after step b) is free of the first solution.

Reaction of Step c:

An embodiment of the present invention relates to the process, wherein the second solution in step c) comprises a base.

An embodiment of the present invention relates to the process, wherein the second solution in step c) comprises a base selected from the group consisting of 4-dimethylaminopyridine (DMAP), 2,3,6,7-tetrahydro-1H,5H-9-azabenzo[ij]quinolizine (psycho-DMAP), 9-azajulolidine (9AJ), 1,1,7,7-Tetramethyl-9-azajulolidine (TMAJ), 4-pyrrolidinopyridine (PPY), 1,4-diazabicyclo[2.2.2]octane (DABCO), 1-methylimidazole (NMI), 1,8-diazabicyclo(5.4.0)undec-7-ene (DBU), 1,5-diazabicyclo(4.3.0)non-5-ene (DBN), triethylamine, N,N-diisopropylethylamine (DIPEA), pyridine, quinuclidine, N,N,N′,N′-tetramethyl guanidin (TMG), 7-methyl-1,5,7-triazabicyclo(4.4.0)dec-5-ene (MTBD), and 1,8-bis(dimethylamino)naphthalene. Preferably bases are 2,3,6,7-tetrahydro-1H,5H-9-azabenzo[ij]quinolizine (psycho-DMAP), 9-azajulolidine (9AJ), 1,1,7,7-Tetramethyl-9-azajulolidine (TMAJ), 4-pyrrolidinopyridine (PPY). The most preferred base is 9-azajulolidine (9AJ). An embodiment of the present invention relates to the process, wherein the second solution in step c) comprises a base at a concentration in the range of 1 mM-500 mM, such as 5 mM-300 mM, such as 5 mM-200 mM, preferably such as 50 mM-200 mM.

An embodiment of the present invention relates to the process, wherein the second solution in step c) comprises a solvent selected from the group consisting of dichloromethane, tetrahydrofuran, toluene, acetonitrile, diethylether, and ethyl acetate.

An embodiment of the present invention relates to the process, wherein the second solution in step c) comprises the second reactant at a concentration in the range of 0.001 mM-500 mM, such as 0.01 mM-400 mM, such as 0.1 mM-300 mM, preferably such as 0.5 mM-200 mM.

An embodiment of the present invention relates to the process, wherein collecting the phosphoramidites in step d) is achieved within a time frame of 1-20 minutes, such as 2-16 minutes, such as 4-10 minutes, preferably such as 6-10 minutes, from the beginning of step c).

An embodiment of the present invention relates to the process, wherein the second solution comprises additives.

### Other Aspects

Another aspect of the present invention relates to an activated resin of a resin, comprising one or more of the heterocyclic moieties selected from the group consisting of imidazole, pyrazole, 1,2,3-triazole, 1,2,4-triazole, tetrazole, 2-nitrophenyltetrazole, 4-nitrophenyltetrazole, 2,4-dinitrophenyltetrazole, 3-nitro-1,2,4-triazole, 4,5-dicyanoimidazole, and 4-nitroimidazole.

An embodiment of the present invention relates to the activated resin, wherein the heterocyclic moieties are in terminal positions. In this context, a terminal position is a position wherein a reactant may react with the heterocyclic moieties without suffering to any particular steric hindrances induced by other parts of the resin.

Yet another aspect of the present invention relates to a loaded resin comprising a resin connected to one or more of the heterocyclic moieties selected from the group consisting of imidazole, pyrazole, 1,2,3-triazole, 1,2,4-triazole, tetrazole, 2-nitrophenyltetrazole, 4-nitrophenyltetrazole, 2,4-dinitrophenyltetrazole, 3-nitro-1,2,4-triazole, 4,5-dicyanoimidazole, and 4-nitroimidazole, wherein each heterocyclic moiety is further connected to a phosphoramidite moiety.

An embodiment of the present invention relates to the loaded resin, wherein the phosphoramidite moieties are in terminal positions. In this context, a terminal position is a position wherein a reactant may react with the phosphoramidite moieties without suffering to any particular steric hindrances induced by other parts of the resin.

An embodiment of the present invention relates to the loaded resin, wherein the phosphoramidite moieties are represented by Formula (8)

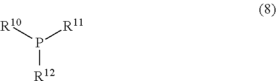

wherein, R¹⁰ is connected to one of the heterocyclic moieties of the loaded resin; R¹¹ is selected from the group consisting of di(C₁-C₆)alkylamino, pyrrolidino, morpholino, bromo, iodo, and chloro; R¹² is selected from the group consisting of (C₁-C₆)alkyl, (C₁-C₆)alkoxy, 2-cyanoethoxy, benzoylthioethylthio, and 1,1-dimethyl-2-cyanoethoxycarbonylmethyl; or R¹¹ and R¹² together with the P to which they are attached form a 1-methyl-1-phenyl-2-oxa-3-phospha-tetrahydropyrrolizine-3-yl radical.

Still another aspect of the present invention relates to a loaded resin obtained by a process comprising the steps of:
a) providing an activated resin of a resin, comprising one or more of the heterocyclic moieties selected from the group consisting of imidazole, pyrazole, 1,2,3-triazole, 1,2,4-triazole, tetrazole, 2-nitrophenyltetrazole, 4-nitrophenyltetrazole, 2,4-dinitrophenyltetrazole, 3-nitro-1,2,4-triazole, 4,5-dicyanoimidazole, and 4-nitroimidazole,
b) obtaining the loaded resin by contacting the activated resin with a first solution comprising a first reactant according to Formula (7)

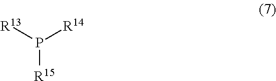

wherein, R¹³ and R¹⁴ are independently selected from the group consisting of di(C₁-C₆)alkylamino, pyrrolidino, morpholino, bromo, iodo, and chloro; R¹⁵ is selected from the group consisting of (C₁-C₆)alkyl, (C₁-C₆)alkoxy, 2-cyanoethoxy, benzoylthioethylthio, and 1,1-dimethyl-2-cyanoethoxycarbonylmethyl, or the first reactant is P-chloro-1-methyl-1-phenyl-2-oxa-3-phospha-tetrahydropyrrolizine.

An embodiment of the present invention relates to the activated resin as described herein, or the loaded resin as described herein, wherein the activated resin or loaded resin is attached to a support selected from the group consisting of a column, tube, pipe, pipette, cylinder, funnel, porous glass, tubular container, needles, beads, pellets, powders, pearls, and grains.

### Alternative Aspects

A first additional aspect of the present invention relates to a process for providing phosphoramidites, which process comprises the steps of:
a) providing an activated resin of a resin, comprising one or more of the heterocyclic moieties selected from the group consisting of imidazole, pyrazole, 1,2,3-triazole, 1,2,4-triazole, tetrazole, 2-nitrophenyltetrazole, 4-nitrophenyltetrazole, 2,4-dinitrophenyltetrazole, 3-nitro-1,2,4-triazole, 4,5-dicyanoimidazole, and 4-nitroimidazole,
b) obtaining a loaded resin by contacting the activated resin with a first solution comprising a first reactant according to Formula (a1)

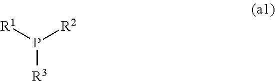

wherein, R¹ and R² are independently selected from the group consisting of di(C₁-C₆)alkylamino, pyrrolidino, morpholino, bromo, iodo, and chloro; R³ is selected from the group consisting of (C₁-C₆)alkyl, (C₁-C₆)alkoxy, 2-cyanoethoxy, benzoylthioethylthio, and 1,1-dimethyl-2-cyanoethoxycarbonylmethyl, or the first reactant is P-chloro-1-methyl-1-phenyl-2-oxa-3-phospha-tetrahydropyrrolizine,
c) contacting a second solution comprising a second reactant with the loaded resin of step b) by flowing the second solution through the loaded resin,
d) collecting the phosphoramidites,

wherein, the second reactant is selected from the group consisting of nucleosides, nucleoside analogs, synthetic nucleosides, disulfides, derivatives of glycols, azides, and ethylene glycol oligomers.

A second additional aspect of the present invention relates to a process for providing phosphoramidites, which process comprises the steps of:
a) providing an activated resin of a resin, according to formula (a2)

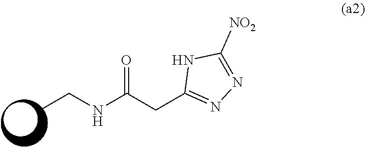

wherein ( [symbol] ) is polystyrene,
b) obtaining a loaded resin by contacting the activated resin with a first solution comprising a first reactant selected from chloro(diisopropylamino)(2-cyanoethoxy)phosphine, bis(diisopropylamino)(2-cyanoethoxy)phosphine, and (1R,7aS)—P-chloro-1-methyl-1-phenyl-2-oxa-3-phospha-tetrahydropyrrolizine
c) contacting a second solution comprising a second reactant with the loaded resin of step b),
d) collecting the phosphoramidites,

wherein, the second reactant is selected from the group consisting of nucleosides, nucleoside analogs, synthetic nucleosides, disulfides, derivatives of glycols, azides, and ethylene glycol oligomers.

A third additional aspect of the present invention relates to a process for providing oligonucleotides, which process comprises the steps of:
a) providing an activated resin of a resin, comprising one or more of the heterocyclic moieties selected from the group consisting of imidazole, pyrazole, 1,2,3-triazole, 1,2,4-triazole, tetrazole, 2-nitrophenyltetrazole, 4-nitrophenyltetrazole, 2,4-dinitrophenyltetrazole, 3-nitro-1,2,4-triazole, 4,5-dicyanoimidazole, and 4-nitroimidazole,
b) obtaining a loaded resin by contacting the activated resin with a first solution comprising a first reactant according to Formula (a3)

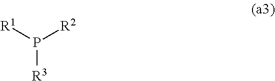

wherein, R¹ and R² are independently selected from the group consisting of di(C₁-C₆)alkylamino, pyrrolidino, morpholino, bromo, iodo, and chloro; R³ is selected from the group consisting of (C₁-C₆)alkyl, (C₁-C₆)alkoxy, 2-cyanoethoxy, benzoylthioethylthio, and 1,1-dimethyl-2-cyanoethoxycarbonylmethyl, or the first reactant is P-chloro-1-methyl-1-phenyl-2-oxa-3-phospha-tetrahydropyrrolizine,
c) contacting a second solution comprising a second reactant with the loaded resin of step b),
d) transferring the produced phosphoramidites directly to automated oligonucleotide synthesis, or concentrating the produced phosphoramidites before transferring to automated oligonucleotide synthesis,
e) collecting the oligonucleotides,

wherein, the second reactant is selected from the group consisting of nucleosides, nucleoside analogs, synthetic nucleosides, disulfides, derivatives of glycols, azides, and ethylene glycol oligomers.

It should be noted that embodiments and features described in the context of one of the aspects of the present invention also apply to the other aspects of the invention. All patent and non-patent references cited in the present application, are hereby incorporated by reference in their entirety.

The invention will now be described in further details in the following non-limiting examples.

### EXAMPLES

### Example 1—Materials and Methods

All chemicals were purchased from Sigma-Aldrich, Carbosynth and Link Technologies Ltd. In Scotland and used without further purification. HPLC grade anhydrous solvents were purchased in Sure/Seal bottles with inert atmosphere or dried prior use by an M-BRAUN solvent purification system. Reactions were monitored by thin-layer chromatography (TLC) on Merck silica 60 F254 plates and visualized by exposure to UV (254 nm) or by staining with solutions of molybdic acid, potassium permanganate, ninhydrin, vanillin, or p-anisaldehyde. Flash column chromatography was performed using Merck silica gel 60 (230-400 mesh) as stationary phase. NMR spectra were recorded on a Bruker BioSpin GmbH Ascend™ 400 and were calibrated using deuterated solvents (deuterated acetonitrile (MeCN), dimethyl sulfoxide (DMSO), chloroform). ¹H NMR was recorded at 400 MHz, ¹³C NMR was recorded at 100 MHz, ¹⁹F NMR was recorded at 376 MHz and ³¹P NMR was recorded at 162 MHz. Chemical shifts are reported in parts per million and following abbreviations were used to explain multiplicities: s=singlet, d=doublet, t=triplet, q=quartet, m=multiplet. Coupling constants are reported in hertz (Hz). HRMS was performed using electrospray ionization on a Bruker Daltonics MicrOTOF.

Oligonucleotides were synthesized in house on a BioAutomation MerMade-12 automated oligonucleotide synthesizer using reagents and preloaded 1000 Å controlled pore glass (CPG) columns purchased from Link Technologies Ltd. in Scotland. Phosphoramidites were synthesized in house or purchased from Link Technologies Ltd. in Scotland and stored under an inert atmosphere at −20° C. until they were used. Oligonucleotide synthesis was carried out under standard conditions unless otherwise stated. The synthesized oligonucleotides were cleaved from solid support using AMA (1:1 40% methylamine/30-33% ammonium hydroxide) or for sequences comprising G, by using concentrated aqueous NH₃ for 30 minutes at 50° C. The mass was confirmed by UHPLC-ESI-TOF on a Shimadzu LCMS-2020 system. All oligonucleotides were HPLC purified on a Hewlett-Packard Agilent Expand C-18 stationary column using the following method (Solvent A: 0.1 M Triethylammonium acetate, pH=7; Solvent B: MeCN; Gradient: 5% to 20% B over 15 minutes, 20% to 70% B 15-20 minutes). The phosphitylating agent (PCI) used in most of the examples was commercially available chloro(diisopropylamino)(2-cyanoethoxy)phosphine. However, synthesis of phosphoramidites using other phosphitylating agents have also been performed.

These phosphitylating agents were obtained from commercial vendor or synthesized as described in Example 3. The substrate alcohols listed in Table 1 were used in the Examples.

TABLE 1
The substrate alcohols.

| | | | Method of |
|---|---|---|---|
| Compound and structure | | Description | provision |
| T-alc | 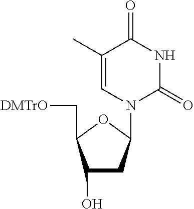 | A nucleoside. Deoxyribose substituted with thymine (T) base and with a hydroxy group protected by DMTr. | Purchased |
| C-alc | 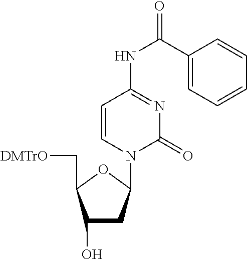 | A nucleoside. Deoxyribose substituted with cytosine (C) base and with a hydroxy group protected by DMTr. An amine group on the base is protected by Bz. | Purchased |
| A-alc | 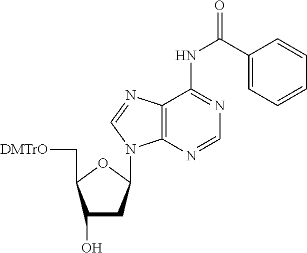 | A nucleoside. Deoxyribose substituted with adenine (A) base and with a hydroxy group protected by DMTr. An amine group on the base is protected by Bz. | Purchased |
| G-alc | 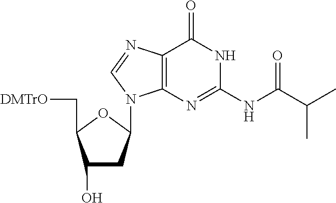 | A nucleoside. Deoxyribose substituted with guanine (G) base and with a hydroxy group protected by DMTr. An amine group on the base is protected by iBu. | Purchased |
| 1-alc | 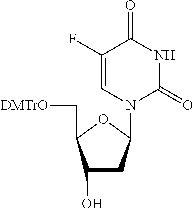 | A nucleoside. Deoxyribose substituted with uracil analog as the base and with a hydroxy group protected by DMTr. | Synthesis in Example 2 |
| 2-alc | 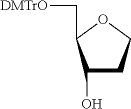 | A nucleoside analog. Tetrahydrofuran without a base and with a hydroxy group protected by DMTr. | Purchased |
| 3-alc | 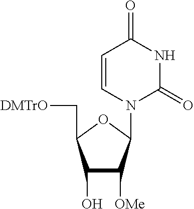 | A nucleoside analog. Ribose substituted with uracil base, methoxy, and with a hydroxy group protected by DMTr. | Purchased |
| 4.1-alc | 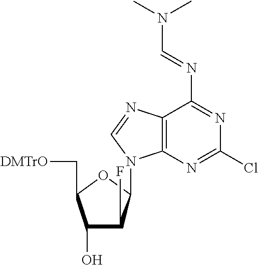 | A nucleoside analog. Ribose substituted with purine analog base, fluoro, and with a hydroxy group protected by DMTr. An amine group on the base has been protected by dmf. | Synthesis in Example 2 |
| 5-alc | 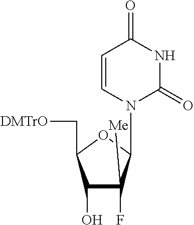 | A nucleoside analog. Ribose substituted with uracil base, methyl, fluoro, and with a hydroxy group protected by DMTr. | Purchased |
| 6-alc | 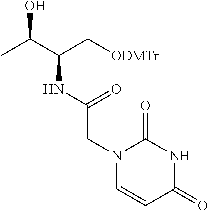 | A synthetic nucleoside. Threoninol which has been backbone modified, substituted with uracil base, and with a hydroxy group protected by DMTr. | Purchased |
| 7-alc | 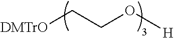 | Ethylene glycol oligomer with a hydroxy group protected by DMTr. | Purchased |
| 8.1-alc | 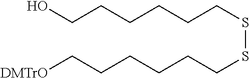 | Bis(6-hydroxyhexyl)disulfide with a hydroxy group protected by DMTr. | Synthesis in Example 2 |

### Example 2—Synthesis of Substrate Alcohols

### Synthesis of 5′-DMTr-Floxuridine (Compound 1-alc)

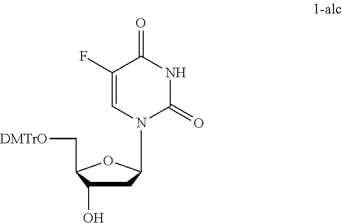

Floxuridine (750 mg, 3.05 mmol, 1.0 eq), 4,4′-dimethoxytrityl chloride (DMTr-Cl) (1.34 g, 3.96 mmol, 1.3 eq) and N,N-dimethylpyridin-4-amine (DMAP) (74 mg, 0.61 mmol, 0.20 eq) were dissolved in pyridine (15 mL) and stirred overnight at room temperature (r.t.). The solvent was then removed under reduced pressure and the residue was redissolved in ethyl acetate (EtOAc) (30 mL) and washed with water (30 mL) and brine (30 mL), dried over Na₂SO₄, filtered and concentrated under reduced pressure. The residue was subjected to flash column chromatography (0-5% MeOH in CH₂Cl₂+1% triethylamine (Et₃N)) to give the desired compound 1-alc (1.256 g, 2.29 mmol, 75%) as a light pink foam. ¹H NMR (400 MHz, CD₃CN) δ_{H} (ppm) 9.27 (broad s, 1H), 7.74 (d, J=6.77 Hz, 1H), 7.43 (d, J=7.20 Hz, 2H), 7.36-7.29 (m, 6H), 7.24 (tt, J=7.18, 2.02 Hz, 1H), 6.86 (dd, J=7.64, 1.27 Hz, 4H), 6.14 (td, J=6.45, 1.67 Hz, 1H), 4.47-4.42 (m, 1H), 3.94 (q, J=4.16 Hz, 1H), 3.77 (s, 6H), 3.39 (broad s, 1H), 3.32 (dd, J=10.74, 4.45 Hz, 1H), 3.24 (dd, J=10.73, 2.97 Hz, 1H), 2.32-2.19 (m, 2H). ¹³C NMR (100 MHz, CD₃CN) δ_{C} (ppm) 159.7, 158.1, 157.9, 149.9, 145.9, 142.7, 140.4, 136.8, 136.6, 131.0, 128.9, 127.9, 125.5, 125.2, 114.1, 87.5, 87.1, 86.1, 71.6, 64.3, 55.9, 41.1. ¹⁹F NMR (376 MHz, CD₃CN) δ_{F} (ppm) −168.43. HRMS (ESI) m/z [M+Na]⁺ calc. for C₃₀H₂₉FN₂O₇ 571.1851, found 571.1852.

### Synthesis of Amine Protected 5′-DMTr-Clofarabine (Compound 4.1-alc)

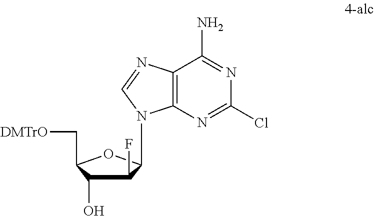

Clofarabine (500 mg, 1.65 mmol, 1.0 eq), DMTr-Cl (642 mg, 1.89 mmol, 1.15 eq) and DMAP (40 mg, 0.33 mmol, 0.20 eq) were dissolved in pyridine (10 mL) and stirred overnight at r.t. The solvent was then removed under reduced pressure and the residue was redissolved in EtOAc (25 mL) and washed with water (25 mL) and brine (25 mL), dried over Na₂SO₄, filtered and concentrated under reduced pressure. The residue was subjected to flash column chromatography (½-1/0 EtOAc/pentane+1% Et₃N) to give compound 4-alc (852 mg, 1.40 mmol, 85%) as a white foam. ¹H NMR (400 MHz, CD₃CN) δ_{H} (ppm) 7.99 (d, J=2.20 Hz, 1H), 7.44 (d, J=7.35 Hz, 2H), 7.35-7.18 (m, 7H), 6.85-6.79 (m, 4H), 6.50 (broad s, 2H), 6.35 (dd, J=15.23, 4.36 Hz, 1H), 5.14 (dt, J=52.23, 4.02 Hz, 1H), 4.55 (d, J=18.56 Hz, 1H), 4.11-4.04 (m, 2H), 3.74 (d, J=1.04 Hz, 6H), 3.44 (dd, J=10.40, 6.45 Hz, 1H), 3.34 (dd, J=10.42, 3.55 Hz, 1H). ¹³C NMR (100 MHz, CD₃CN) δ_{C} (ppm) 159.6, 157.7, 154.8, 151.8, 146.0, 141.4, 141.3, 136.8, 136.8, 131.0, 131.0, 129.0, 128.8, 127.8, 119.0, 114.0, 97.3, 95.3, 87.1, 83.5, 83.4, 83.3, 83.1, 75.2, 74.9, 64.2, 55.9. ¹⁹F NMR (376 MHz, CD₃CN) δ_{F} (ppm) −199.04. HRMS (ESI) m/z [M+H]⁺ calc. for C₃₁H₂₉ClFN₅O₅ 606.1914, found 606.1923.

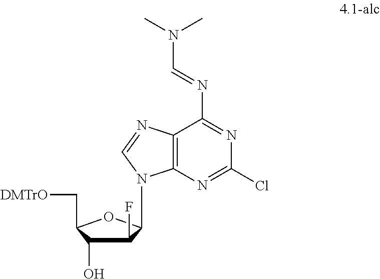

Compound 4-alc (780 mg, 1.29 mmol, 1.0 eq) was dissolved in dry MeOH (20 mL) and N,N-dimethylformamide dimethyl acetal (0.857 mL, 6.44 mmol, 5.0 eq) was added and the mixture was stirred overnight at r.t. The mixture was then diluted with EtOAc (50 mL) and washed 5 times with water (50 mL), brine (50 mL), dried over Na₂SO₄, filtered and concentrated under reduced pressure to give the desired compound 4.1-alc (851 mg, 1.29 mmol, quant.) as a white foam. ¹H NMR (400 MHz, CD₃CN) δ_{H} (ppm) 8.87 (s, 1H), 8.03 (d, J=2.32 Hz, 1H), 7.43 (d, J=7.11 Hz, 2H), 7.35-7.19 (m, 7H), 6.85-6.80 (m, 4H), 6.38 (dd, J=15.42, 4.37 Hz, 1H), 5.16 (dt, J=52.07 Hz 4.28 Hz, 1H), 4.57 (d, J=18.55 Hz, 1H), 4.12-4.06 (m, 2H), 3.75 (d, J=1.60 Hz, 6H), 3.42 (dd, J=10.43, 6.42 Hz, 1H) 3.33 (dd, J=10.87, 3.53 Hz, 1H), 3.19 (s, 3H), 3.16 (s, 3H). ¹³C NMR (100 MHz, CD₃CN) δ_{C} (ppm) 161.7, 159.7, 159.7, 154.3, 153.7, 146.0, 142.3, 142.3, 136.8, 136.8, 131.0, 131.0, 129.0, 128.8, 127.9, 125.5, 114.0, 97.3, 95.4, 87.1, 83.5, 83.5, 83.2, 83.1, 75.2, 75.0, 64.2, 55.9, 41.8, 35.5. ¹⁹F NMR (376 MHz, CD₃CN) δ_{F} (ppm) −199.0. HRMS (ESI) m/z [M+H]⁺ calc. for C₃₄H₃₄ClFN₆O₅ 661.2336, found 661.2339.

### Synthesis of Disulfide (Compound 8.1-alc)

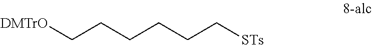

1,6-Hexanediol (17.4 g, 148 mmol, 10 eq) and triethylamine (2.26 mL, 16.2 mmol, 1.1 eq) were dissolved in tetrahydrofuran (THF) (100 mL) and DMTr-Cl (5.00 g, 14.8 mmol, 1.0 eq) was added. The mixture was stirred at r.t. overnight. diethyl ether (Et₂O) (200 mL) was added and the organic phase was washed 3 times with water (100 mL) and once with brine (100 mL), dried over Na₂SO₄, filtered and concentrated under reduced pressure. The residue was dissolved in CH₂Cl₂ (50 mL) and cooled in an ice bath. N-Methyl morpholine (5.67 mL, 51.6 mmol, 3.5 eq) was added along with methanesulfonyl chloride (MsCl) (1.37 mL, 17.7 mmol, 1.2 eq) and the mixture was stirred at 0° C. for 30 min. after which the reaction mixture was allowed to warm to r.t. and stirred overnight. The reaction was quenched with water (50 mL) and the phases were separated. The organic phase was washed twice with water (50 mL), once with brine (50 mL) and then dried over Na₂SO₄, filtered and concentrated under reduced pressure. The residue was dissolved in MeCN (50 mL) along with potassium p-toluenethiosulfonate (4.12 g, 18.2 mmol, 1.23 eq) and the mixture was stirred at 75° C. overnight. The mixture was then diluted with Et₂O and washed with water and brine, dried over Na₂SO₄, filtered and concentrated under reduced pressure. The residue was purified with flash column chromatography (3/1-0/1 pentane/CH₂Cl₂ gradient+1% Et₃N) to give compound 8-alc (5.507 g, 9.32 mmol, 63% for 3 steps) as a clear oil. ¹H NMR (400 MHz, CDCl₃) δ_{H} (ppm) 7.77 (d, J=8.29 Hz, 2H), 7.40 (d, J=7.20 Hz, 2H), 7.32-7.23 (m, 8H), 7.18 (t, J=7.20 Hz, 1H), 6.82 (d, J=8.84 Hz, 4H), 3.77 (s, 6H), 2.99 (t, J=6.43 Hz, 2H), 2.94 (t, J=7.38 Hz, 2H), 2.41 (s, 3H), 1.59-1.48 (m, 4H), 1.33-1.19 (m, 4H). ¹³C NMR (100 MHz, CDCl₃) δ_{C} (ppm) 158.5, 145.5, 144.7, 142.3, 136.8, 130.1, 129.9, 128.3, 127.8, 127.1, 126.7, 113.1, 85.8, 63.2, 55.3, 36.1, 29.9, 28.7, 28.5, 25.8, 21.7. HRMS (ESI) m/z [M−K]⁺ calc. for C₃₄H₃₈O₅S₂ 629.1792, found 629.1785.

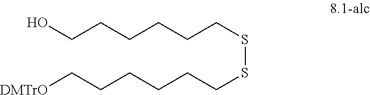

Compound 8-alc (484 mg, 819 μmol, 1.1 eq) was dissolved in CH₂Cl₂ (10 mL) and triethylamine (156 μL, 1.12 mmol, 1.5 eq) was added. 6-Mercapto-1-hexanol (102 μL, 745 μmol, 1.0 eq) was added and the mixture was stirred for 30 min. at r.t. The solvent was then removed under reduced pressure and the residue was subjected to flash column chromatography (1/3-1/0 Et₂O/pentane+1% Et₃N) to give the desired compound 8.1-alc (396 mg, 693 μmol, 93%) as a clear oil. ¹H NMR (400 MHz, CD₃CN) δ_{H} (ppm) 7.42 (d, J=7.38 Hz, 2H), 7.32-7.26 (m, 6H), 7.21 (t, J=7.21 Hz, 1H), 6.86 (d, J=8.86 Hz, 4H), 3.76 (s, 6H), 3.45 (q, J=5.50 Hz, 2H), 3.00 (t, J=6.46 Hz, 2H), 2.68 (q, J=7.45 Hz, 4H), 2.46 (t, J=5.33 Hz, 1H), 1.69-1.54 (m, 6H), 1.50-1.43 (m, 2H), 1.40-1.27 (m, 8H). ¹³C NMR (100 MHz, CD₃CN) δ_{C} (ppm) 159.5, 146.7, 137.6, 130.9, 129.0, 128.7, 127.6, 113.9, 86.5, 64.0, 62.5, 55.9, 39.5, 33.5, 30.5, 29.9, 29.8, 29.0, 28.8, 26.6, 26.2. HRMS (ESI) m/z [M+H]⁺ calc. for C₃₃H₄₄O₄S₂ 591.2573, found 591.2570.

### Example 3—Synthesis of Phosphitylating Agents

### Synthesis of (1R,7caS)—P-chloro-1-methyl-1-phenyl-2-oxa-3-phospha-tetrahydropyrrolizine

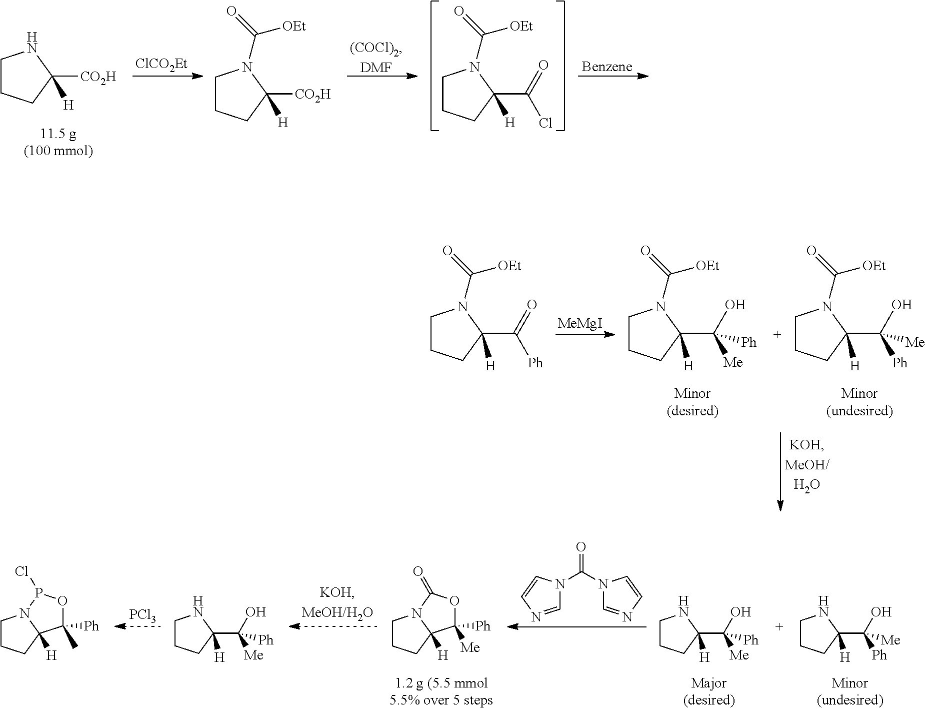

The synthesis is described further in Angewandte Chemie International Edition 48.3 (2009): 496-499.

### Example 4—On-Demand Synthesis of Phosphoramidites

System Setup

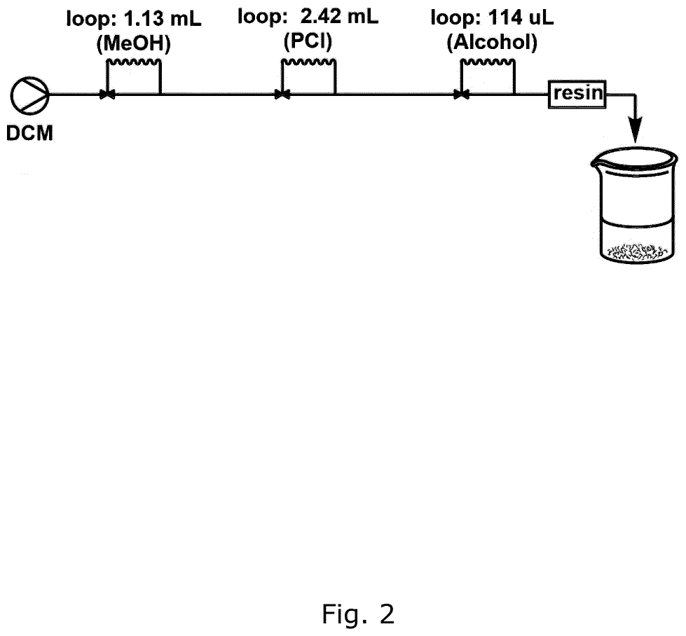

The tubing throughout the system contained of stainless steel tubing ( 1/16″ outside diameter (OD)×0.75 mm inside diameter (ID)) and connections were made with PEEK or stainless steel HPLC fittings (all with 1/16″ ID). A HPLC pump (Knauer Azura P 4.1S) was used to pump CH₂Cl₂ through the reactor system that consisted of one backpressure regulator, three injections valves (2 position: load and inject, 6-port, 1/16″, Vici) in series, and a column packed with an activated resin. The exiting fluid was collected in an 8 mL vial under argon atmosphere, unless otherwise stated. A schematic representation of the system is depicted in FIG. 2.

Procedure

The phosphoramidites in the residence time study were prepared by the following procedure:
i) An activated resin obtained in Example 8 (AM-PS-Het5, 250 mg) was loaded into an HPLC column of stainless steel (75 mm×4.6 mm). The resulting column packed with the activated resin was flushed with toluene at a flow rate of 1.00 mL/min for 15 minutes. The column was weighed (m_{Toluene+resin+column}=43.118 g), flushed with dichloromethane (DCM) with a flow rate of 1.00 mL/min for 15 minutes, and then weighted again (m_{DCM+resin+column}=43.588 g). The void volume, V_{void}, of the system was determined to establish a connection between flow rate and estimated residence time of the liquid passing through the column. The void volume of the column was calculated using the formula:

$${V}_{void} = \frac{Δ {m}_{solvent switch}}{Δ {ρ}_{solvent}} = \frac{43.5588 g - 43.118 g}{1.33 \frac{g}{mL} - 0.867 \frac{g}{mL}} = 1. mL$$

wherein, Δm_{solvent switch} is the difference in mass and Δρₛₒₗᵥₑₙₜ the difference in density when switching solvents,
ii) a loaded resin was prepared by loading the activated resin 4 times with PCI (0.10 M) and N,N-diisopropylethylamine (DIPEA, 0.10 M) in DCM (2 mL) with flow rate of 1.00 mL/min for 5 minutes including DCM wash,
iii) the substrate alcohol (0.10 M, 0.114 mmol) and DMAP (0.15 M, 0.171 mmol) or another base, such as PPY or 9AJ, were dissolved in DCM (1 mL) and eluted through the loaded resin with a flow rate of between 0.125 mL/min and 1.00 mL/min (residence time between 8 minutes and 1 minute),
iV) fractions comprising the synthesized phosphoramidites were collected for 2.5 times the residence time.

A new synthesis using another substrate alcohol could be performed simply by reloading the already used column with a solution of PCI (0.10 M, 0.20 mmol) and DIPEA (0.15 M, 0.30 mmol) in DCM (2 mL) at a flow rate of 1.00 mL/min for 5 minutes, and then followed by a DCM wash with a flow rate of 1.00 mL/min for 5 minutes.

Results

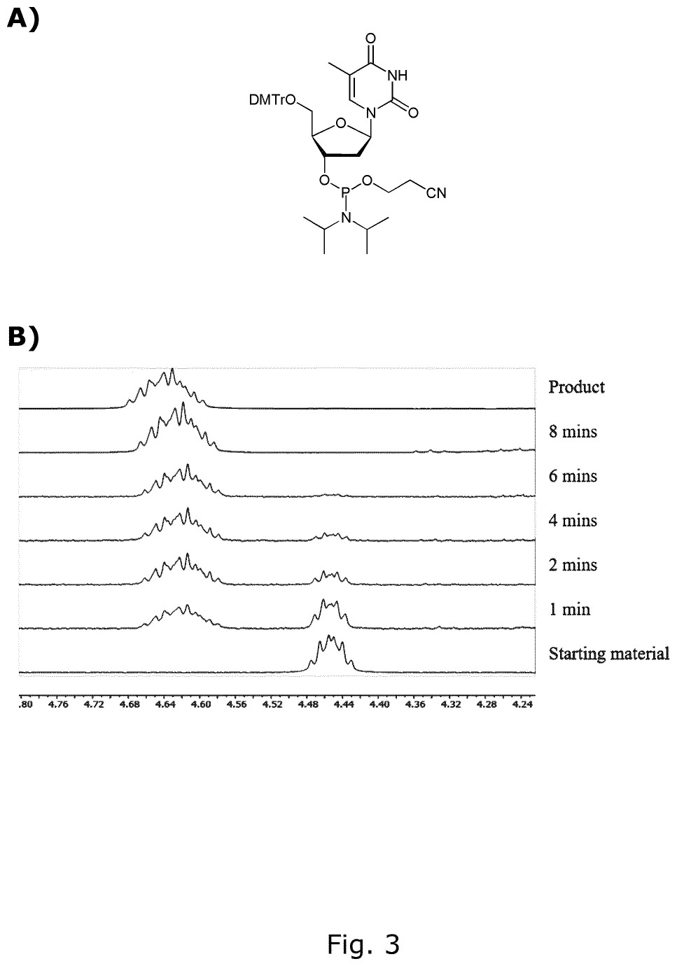

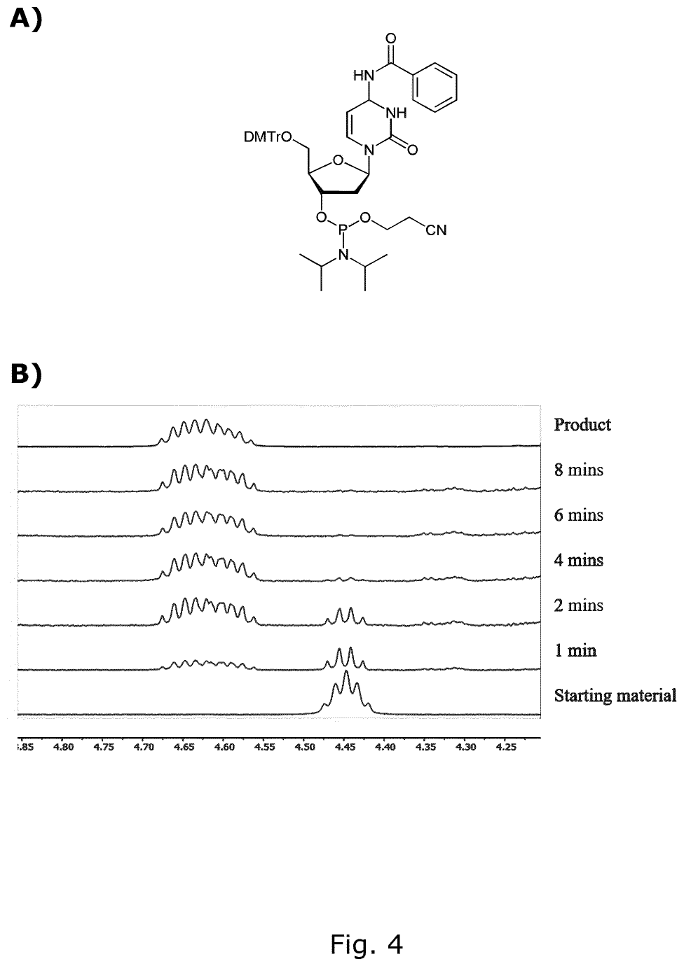

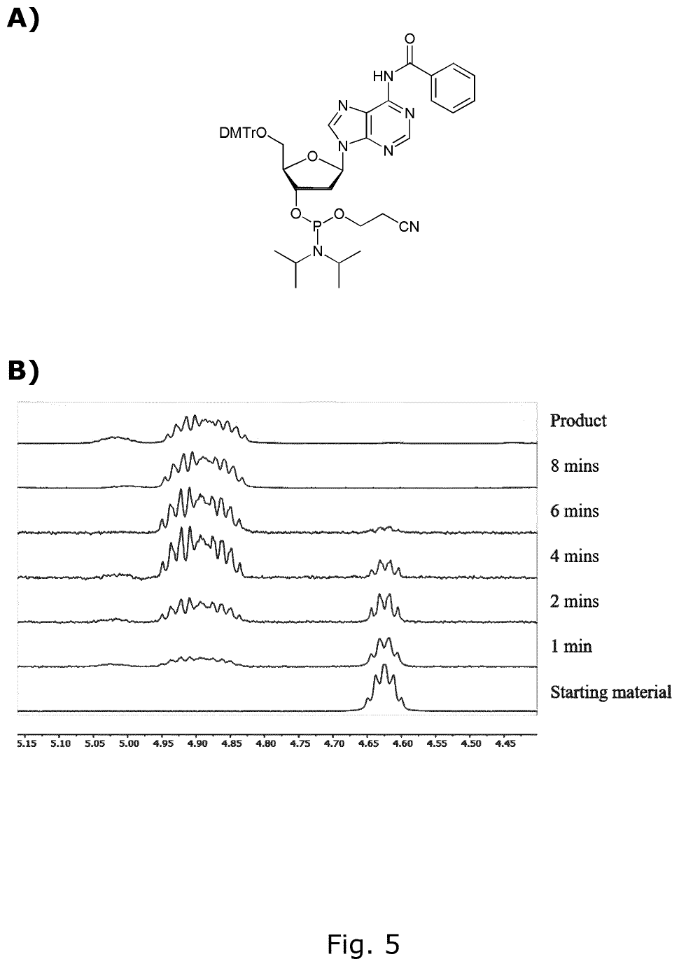

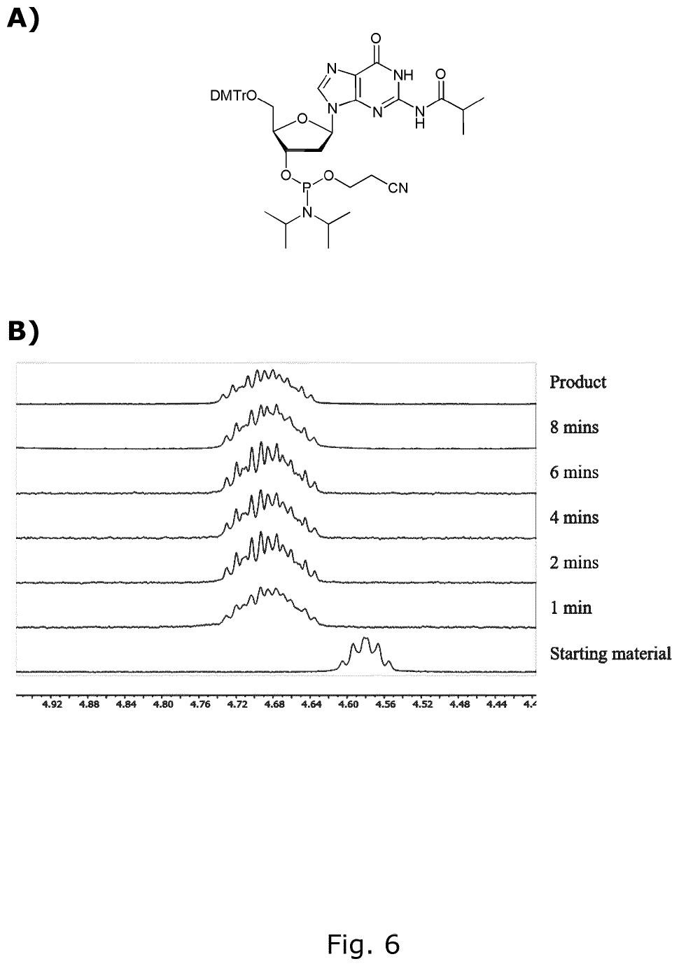

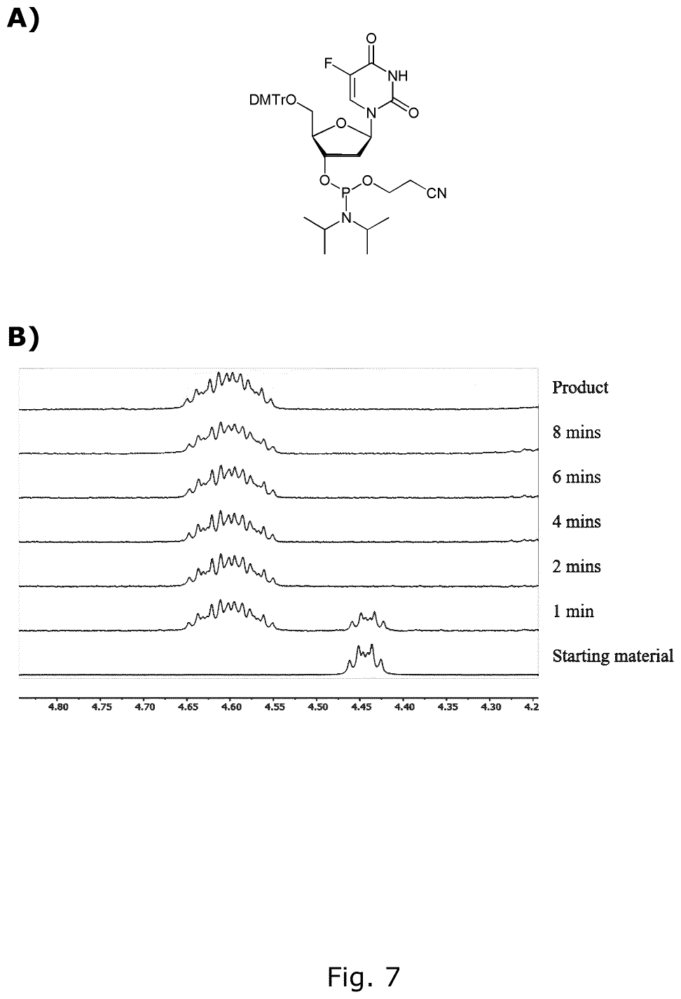

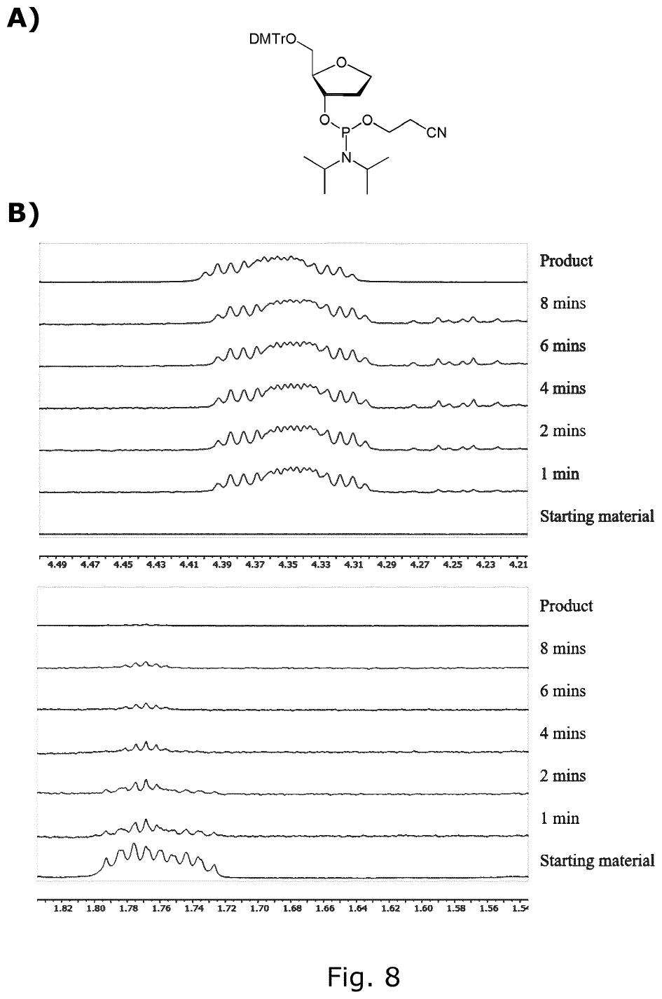

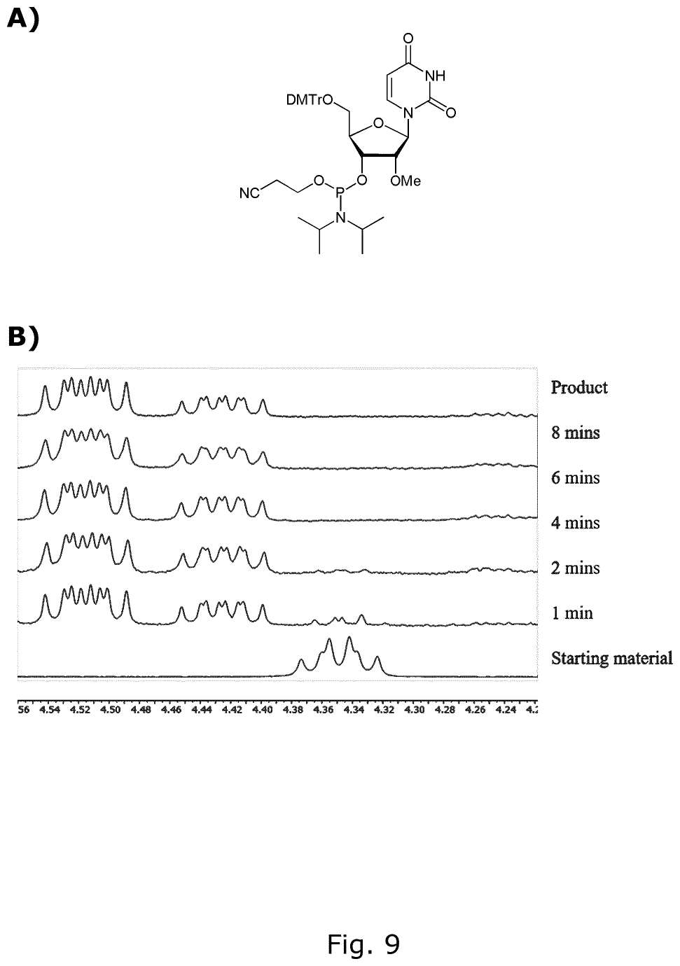

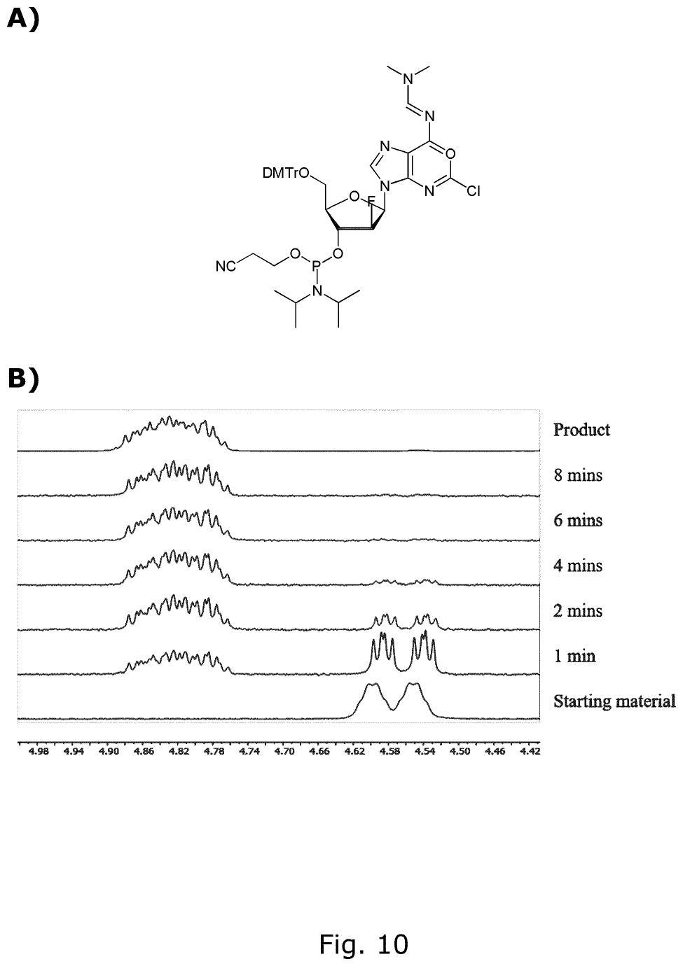

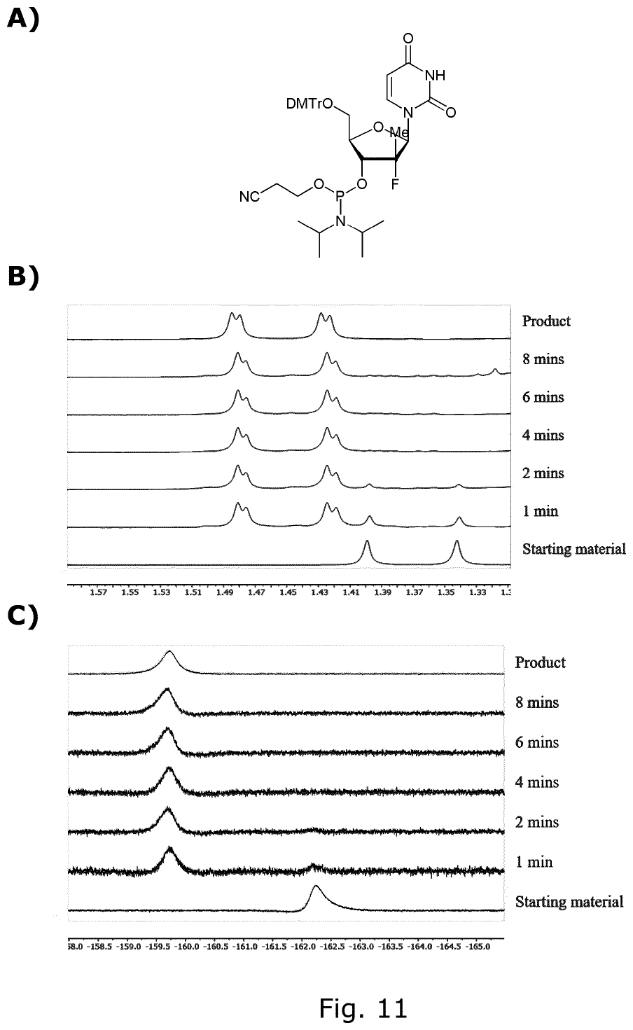

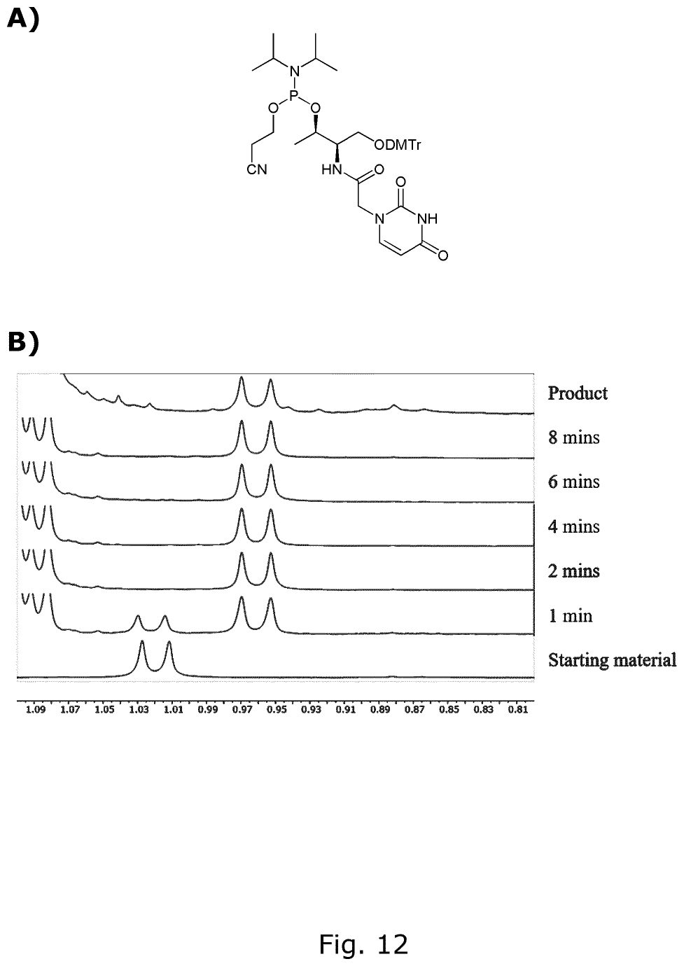

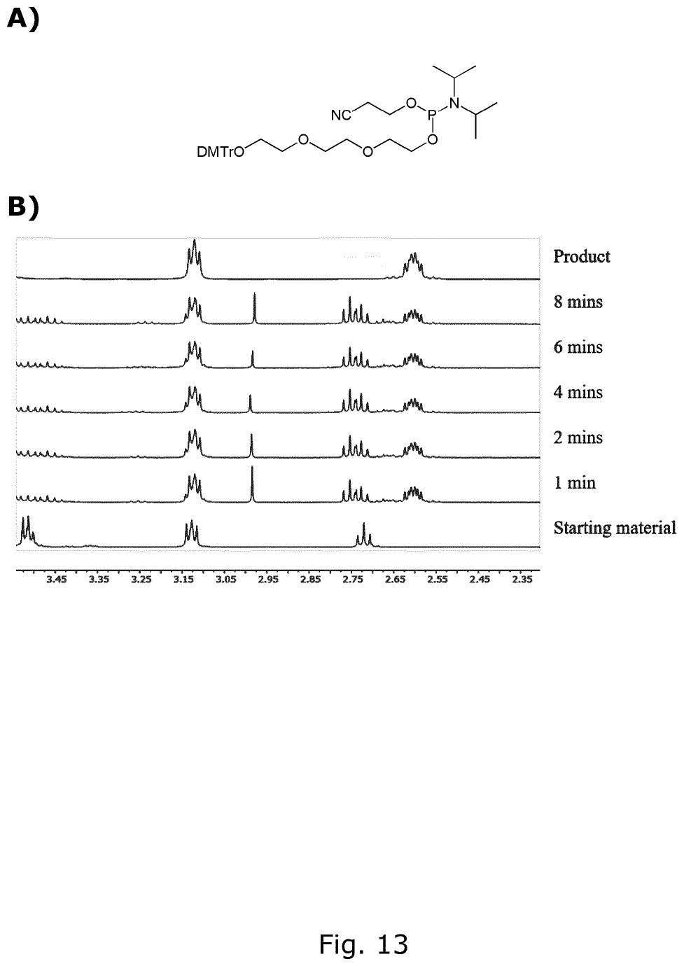

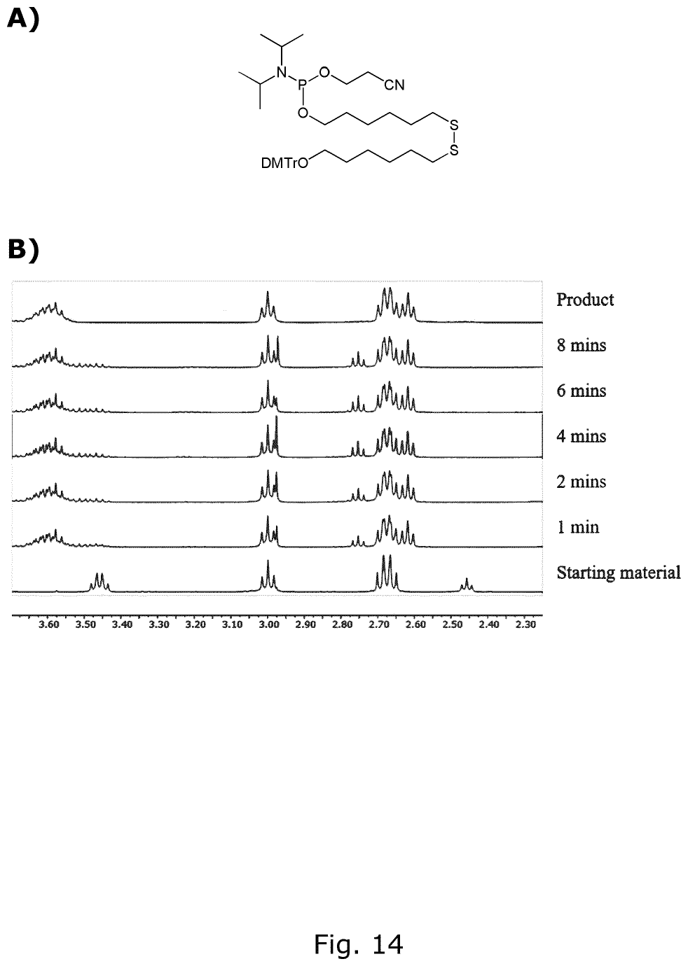

The collected fractions were concentrated under reduced pressure and analyzed by ¹H and ¹⁹F NMR. The yields were calculated by ¹H NMR integration of non-overlapping signals between starting material and product. The results obtained using the PCI, chloro(diisopropylamino)(2-cyanoethoxy)phosphine, and the base, DMAP, are given in the Table 2a. The structures of the products are shown in FIGS. 3 to 14 as inserts A). Inserts B), show ¹H NMR spectra of characteristic peaks of the products and starting materials in the samples obtained for each residence time experiment, for the pure starting materials, and for the pure reference phosphoramidites. FIGS. 11, provides an additional insert C) showing ¹⁹F NMR spectra of characteristic peaks.

TABLE 2a
Results of the on-demand synthesis.

| | Optimal | | | | | | Phosphor- |
|---|---|---|---|---|---|---|---|
| Substrate | flow rate | Residence time and yield | | | | | amidite |
| Alcohol | (ml/min) | 1 min | 2 min | 4 min | 6 min | 8 min | produced |
| T-alc | 0.125 | 58.2% | 80.4% | 88.2% | 96.3% | 100% | T |
| C-alc | 0.167 | 59.1% | 84.7% | 97.6% | 100% | 100% | C |
| A-alc | 0.125 | 44.2% | 67.7% | 88.7% | 94.9% | 100% | A |
| G-alc | 1.000 | 100% | 100% | 100% | 100% | 100% | G |
| 1-alc | 0.500 | 79% | 100% | 100% | 100% | 100% | 1 |
| 2-alc | 0.250 | 89% | 91% | 100% | 100% | 100% | 2 |
| 3-alc | 0.250 | 95% | 99% | 100% | 100% | 100% | 3 |
| 4.1-alc | 0.167 | 50% | 81% | 93% | 100% | 100% | 4.1 |
| 5-alc | 0.250 | 86% | 95% | 100% | 100% | 100% | 5 |
| 6-alc | 0.500 | 67% | 100% | 100% | 100% | 100% | 6 |
| 7-alc | 1.000 | 100% | 100% | 100% | 100% | 100% | 7 |
| 8.1-alc | 1.000 | 100% | 100% | 100% | 100% | 100% | 8.1 |

It should be noted that for phosphoramidite product 3 the integration of ¹³CHD₂CN overlaps with the starting material giving 0.55% additional integral of the residual solvent peak. This was subtracted.

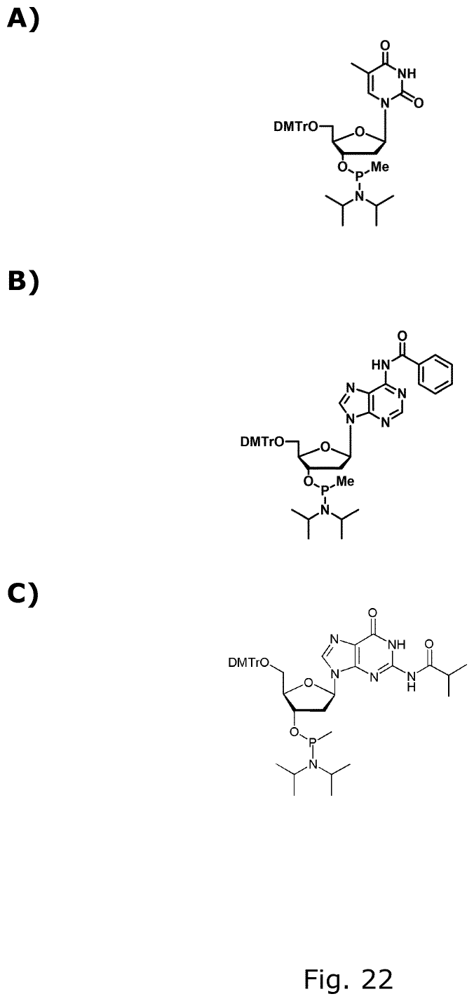

Synthesis of phosphoramidites was also performed using the PCI, chloro(diisopropylamino)(methyl)phosphine using 9AJ as base, and the results are shown in Table 2b. The structures of the products are shown in FIGS. 22A-22C.

TABLE 2b
Results of the on-demand synthesis.

| | Base | | | | | Phosphor- |
|---|---|---|---|---|---|---|
| Substrate | for | Residence time and yield | | | | amidite |
| Alcohol | elution | 2 min | 5 min | 10 min | 15 min | produced |
| T-Alc | 9AJ | 80% | 100% | — | — | 9 |
| A-Alc | 9AJ | 23% | 61% | 89% | 91% | 10 |
| G-Alc | 9AJ | 91% | 94% | 92% | — | 11 |

The amount concentration of 9AJ was 300 mM.

Conclusion: The Example shows that the on-demand synthesis is very efficient with both DMAP and 9AJ and may produce phosphoramidites of various types within a few minutes. Comparison shows that 9AJ is even more efficient than DMAP as base.

### Example 5—Generalized Procedure for Synthesis of Oligonucleotides from On-Demand Synthesized Phosphoramidites

On-Demand Synthesis of Phosphoramidites

A system setup similar to that described in Example 4 was used. An activated resin obtained in Example 8 (AM-PS-Het5, 250 mg) was loaded into a of stainless steel HPLC column (75 mm×4.6 mm). The activated resin was initially loaded 4 times with PCI (0.10 M) and DIPEA (0.10 M) in CH₂Cl₂ (2 mL) with flow rate=1.00 mL/min for 5 minutes and then followed by a DCM wash with a flow rate of 1.00 mL/min for 5 minutes. The following procedure was then used when performing the phosphoramidite synthesis:
a substrate alcohol (0.10 M) and DMAP (0.15 M) in DCM (0.114 mL) were eluted through the loaded resin with a flow rate equal to the optimal flow rate found in Example 4 for synthesis using each of the substrate alcohols,
fractions comprising the synthesized phosphoramidites were collected for 2.5 times the residence time.

A new synthesis using another substrate alcohol could be performed simply by reloading the already used column with a solution of PCI (0.10 M) and DIPEA (0.15 M) in DCM (2 mL) once with a flow rate of 1.00 mL/min for 5 minutes (including CH₂Cl₂ wash).

Oligonucleotide Synthesis

The collected fractions comprising the synthesized phosphoramidites were concentrated under reduced pressure and redissolved in acetonitrile (MeCN, 100 μL per fraction). Then, the fractions were applied to automated oligonucleotide synthesis (ON) on controlled pore glass (CPG). The coupling conditions are summarized in Table 3.

TABLE 3
Coupling conditions for automated ON synthesis.

| Reaction | | |
|---|---|---|
| step | Reagents | Volume |
| Wash | MeCN | 150 μL |
| Deblock | 3% TCA in CH₂Cl₂ (w/v) | 150 μL |
| Coupling | 0.1M phosphoramidite, 0.25M ETT (1/1, v/v) | 140 μL |
| Oxidation | 0.02M I₂, TPF/pyridine/H₂O (7/2/1, v/v) | 150 μL |
| Capping | THF/Ac₂O/NMI (18/1/1, v/v) | 150 μL |

Coupling times of 2×60 s, 2×90 s, or 2×360 s were used (see specific Examples for further details). During coupling, 50 μL of a phosphoramidite in MeCN was mixed with 90 μL 5-(ethylthio)-1H-tetrazole (ETT, 0.5 M) in MeCN.

After synthesis each oligonucleotide was still attached to the controlled pore glass. To obtain the free oligonucleotides, any sequences comprising G were cleaved from the CPG by treatment with AMA for 30 minutes at 65° C. The other sequences were cleaved by treatment with concentrated aqueous NH₃ for 30 minutes at 50° C. The supernatant was concentrated under reduced pressure and the residue subjected to HPLC purification. The conditions applied for cleaving the oligonucleotides from the controlled pore glass also changes specific chemical groups in the oligonucleotide sequence to amino or hydroxy groups, whereby protecting groups are removed.

The yields were determined by measuring the UV absorbance at 260 nm of the collected samples. HPLC purification and analysis could be performed using the following methods:

### Example 6—Synthesis of Single Coupled Oligonucleotides

Synthesis of the phosphoramidites and the subsequent coupling to oligonucleotides was performed according to the generalized procedures described in Example 5. The coupling time used to prepare each of the oligonucleotides are given in Table 4.

The prepared single coupled oligonucleotides were in accordance with the sequence: (T₇XT₇): 5′TTT TTT TXT TTT TTT 3′

wherein, X refers to a phosphoramidite prepared by the on-demand synthesis and which has been coupled to take part in the sequence as an oligonucleotide residue. Each T refers to a residue of a thymidine phosphoramidite obtained from a commercial vendor.

Yields were determined by measuring UV-analysis of the collected fractions. Except for compound 8.1, for which the oligonucleotide synthesis was stopped after coupling with 8.1, the coupling yield was based on integration ratio of absorbance at 260 nm between the truncated oligonucleotide (T₇): 5′ TTT TTT T 3′ and the desired full-length oligonucleotide (T₇XT₇) by the formula:

$$Yield = \frac{{A}_{{T}_{7} {XT}_{7}}}{{A}_{Total}} = \frac{{A}_{{T}_{7} {XT}_{7}}}{{A}_{{T}_{7} {XT}_{7}} + \frac{{ε ( {T}_{7} {XT}_{7} )}_{260}}{{ε ( {T}_{7} )}_{260}} \cdot {A}_{{T}_{7}}}$$

wherein, ε is the molar extinction coefficient for the given oligonucleotide or truncated oligonucleotide. The molar extinction coefficients were calculated by use of the Molbiotools DNA calculator 2020. For the nucleotides 1, 2, 4 and 6, thymidine was used in the calculation of E and for 5.1, adenosine was used instead. For the calculation of oligonucleotides containing 3, 7 and 8.1, ε was calculated as 2 times ε(T₇). The yield of the 8.1 sequence was determined by HPLC analysis.

The results are summarized in Table 4 and shows coupling yields in the range of 86.7% to 99.6%.

Conclusion: The Examples show that various single coupled oligonucleotides can be created by a combined process wherein the phosphoramidites for automated ON synthesis are provided directly by the on-demand method without any need of purifying the phosphoramidites.

TABLE 4
Results for the single coupled oligonucleotides.

| X in sequence | Coupling time | Coupling yield |
|---|---|---|
| T | 2 × 90 s | 99.2% |
| A | 2 × 90 s | 99.5% |
| C | 2 × 90 s | 99.6% |
| G | 2 × 90 s | 98.3% |
| 1 | 2 × 90 s | 99.2% |
| 2 | 2 × 90 s | 86.7% |
| 3 | 2 × 90 s | 97.5% |
| 4 | 2 × 90 s | 97.0% |
| 5 | 2 × 360 s | 98.7% |
| 6 | 2 × 360 s | 89.8% |
| 7 | 2 × 90 s | 95.8% |
| 8.1 | 2 × 90 s | 96.8% |

### Example 7—Synthesis of Complex Oligonucleotides

Synthesis of the phosphoramidites and the subsequent coupling to oligonucleotides was performed according to the generalized procedures described in Example 5. Reference oligonucleotides were prepared from reference phosphoramidites, which were purchased from Link Technologies Ltd in Scotland and stored under an inert atmosphere at −20° C. until they were used. The coupling times for preparation of each of the oligonucleotides are given in Tables 5, 6 and 7.

Oligonucleotides of the following sequences were synthesized:

Sequence 1 (10-mer):
5′ TTT TTT TTT T 3′
Sequence 2 (13-mer):
5′ TAC GTG ACC TGA T3′
Sequence 3 (51-mer):
5′ CCG CTT TCT AGT TCG
TCC TCC ATA ATT AAT TTC
CTA GAG TCC TAC GTG CTC 3′.

The obtained oligonucleotides were then purified and analysed using the following HPLC methods:

Method 1:
Solvent A: 0.1 M Triethylammonium acetate, pH=7
Solvent B: MeCN
Gradient: 5% to 20% B over 15 minutes, 20% to 70% B 15-20 minutes

Method 2:
Solvent A: 0.1 M Triethylammonium acetate, pH=7
Solvent B: MeCN
Gradient: 5% to 15% B over 15 minutes, 20% to 70% B 15-20 minutes

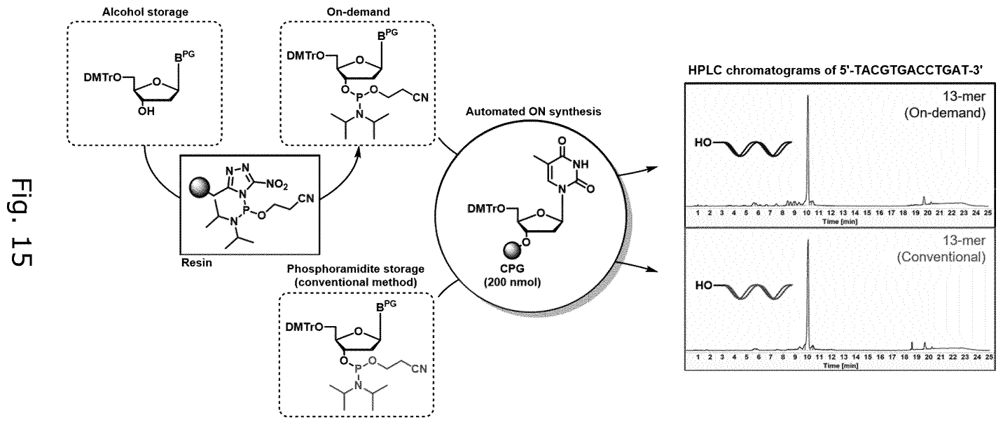

FIG. 15 shows that the 13-mer oligonucleotide prepared from on-demand synthesized phosphoramidites may be obtained at a purity measured by HPLC, which is at least similar to the purity of the reference oligonucleotide. Further details for comparison between the produced oligonucleotides are listed in Tables 5, 6 and 7:

TABLE 5
Results for sequence 1.

| Coupling time | ONs from reference | ONs from on-demand |
|---|---|---|
| 2 × 90 s | phosphoramidites | synthesized phosphoramidites |
| Calc. mass | 2979.9839 | 2979.9839 |
| LCMS found | 2979.9787 | 2979.9787 |
| Average | 96.7% | 93.6% |
| Coupling Yield | | |

HPLC method 1, Coupling time 2 × 90 s,

TABLE 6
Results for sequence 2.

| Coupling time | ONs from reference | ONs from on-demand |
|---|---|---|
| 2 × 90 s | phosphoramidites | synthesized phosphoramidites |
| Calc. mass | 3949.6221 | 3949.6221 |
| LCMS found | 3949.3250 | 3949.3250 |
| Average | 95.9% | 92.0% |
| Coupling Yield | | |

HPLC method 1, Coupling time 2 × 90 s,

TABLE 7
Results for sequence 3.

| Coupling time | ONs from reference | ONs from on-demand |
|---|---|---|
| 2 × 60 s | phosphoramidites | synthesized phosphoramidites |
| Calc. mass | 15467.92 | 15467.92 |
| LCMS found | 15467.62 | 15467.64 |
| Average | 98.3% | 96.1% |
| Coupling Yield | | |

HPLC method 2, Coupling time 2 × 60 s,

Conclusion: The Examples shows that complex oligonucleotides can be created by a combined process wherein the phosphoramidites for automated ON synthesis are provided directly by the on-demand method without any need of purifying the phosphoramidites. The produced oligonucleotides are high purity products which are considered ready for use in PCR or similar technology.

### Example 8—Activation of Resins

Resins:

The resins purchased from commercial vendors and used to create the activated resins are:

TentaGel™ S—NH₂ (TG):
Main material: Polystyrene (PS) with polyethylene glycol (PEG).
   Description: Grafted copolymer of cross-linked PS ( [symbol] ) with PEG grafts and terminal amino groups.

[symbol]

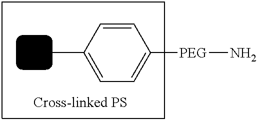

Supplier: Sigma-Aldrich

(Aminomethyl)polystyrene (AM-PS):
Main material: (Aminomethyl)polystyrene
   Description: Cross-linked PS ( [symbol] ) with terminal aminomethyl (AM) groups

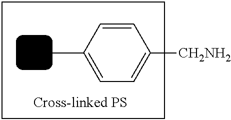

Supplier: Sigma-Aldrich

Amino-SynBase™ Controlled Pore Glass 3000/110 (LCAA):
Main material: Silica
   Description: Porous glass
      Formula: SiO₂
      Supplier: LinkTeck

PEGA
Main material: Polyacrylamide with PEG branching
   Supplier: Sigma-Aldrich

Aminomethyl ChemMatrix® (AM-CM):
Main material: Polyethylene glycol (PEG)
   Supplier: Sigma-Aldrich

TentaGel® XV HMPA (TG-XV):
Main material: Polystyrene (PS) with polyethylene glycol (PEG).
   Supplier: Rapp-polymere

HypoGel® Resin (HypoGel):
Main material: Polystyrene (PS) with polyethylene glycol (PEG) branching.
   Supplier: Rapp-polymere

Aminobutyl Polystyrene (AB-PS):
Main material: Polystyrene (PS)
   Supplier: Rapp-polymere

Functionalized Heterocycles for Activation of Resins:

The functionalized heterocycles used to create the activated resins are listed in Table 8, most were purchased from commercial vendors and are referred to as:

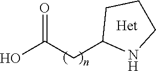

wherein,

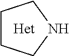

is a heterocycle of 1,2,3-triazole, 1,2,4-triazole, tetrazole, 3-nitro-1,2,4-triazole, or imidazole. The commercial vendors were Sigma-Aldrich or Alfa Aesar.

TABLE 8
The functionalized Heterocycles.

| | | Method of |
|---|---|---|
| Abb. | Structure and name | provision |
| Het1 | 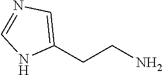 | Purchased |
| | 2-(imidazol-4-yl)ethylamine | |
| Het2 | 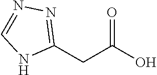 | Purchased |
| | 2-(4H-1,2,4-triazol-3-yl)acetic acid | |
| Het3 | 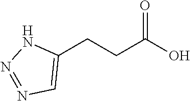 | Synthesized according to reference ^{a} |
| | 3-(1H-1,2,3-Triazol-4-yl)propanoic acid | |
| Het4 | 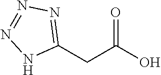 | Purchased |
| | 2-(1H-tetrazol-5-yl)acetic acid | |
| Het5 | 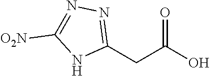 | Synthesized according to reference ^{b} |
| | 2-(5-Nitro-4H-1,2,4-triazol-3-yl)acetic acid | |

^{a} Het3 was synthesized according to previously published procedure by C. G. Thomson et al., Bioorg. Med. Chem. Lett., 2018, 28, 2279-2284.
^{b} Het5 was synthesized according to previously published procedure by V. Thottempudi and J. M. Shreeve, J. Am. Chem. Soc., 2011, 133, 19982-19992.

Activation of the Resins:

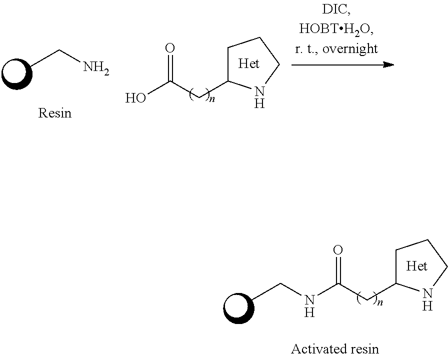

General Procedure for Coupling Between Amine-Functionalized Resins and Carboxylic Acids:

The carboxylic acid was dissolved in CH₂Cl₂ (0.205 M, 10 equivalent(eq)) and N,N′-diisopropylcarbodiimide (DIC, 0.205 M, 10 eq), DIPEA (0.615 M, 30 eq) and 1-hydroxybenzotriazole hydrate (HOBt·H₂O, 0.205 M, 10 eq) were added. The mixture was stirred at r.t. for 20 minutes and then added to the amine-functionalized resin (1 eq) in a plastic column (PD-10 from GE Lifesciences) and shaken overnight at r.t. The resin was then washed with MeOH, CH₂Cl₂ and Et₂O.

The beads were analysed by Kaiser test which gave negative results for amines meaning quantitative coupling yields.

This procedure was performed for the following combinations of resins and functionalized heterocycles:
TentaGel™ S—NH₂ (TG) was functionalized with
2-(4H-1,2,4-triazol-3-yl)acetic acid (TG-Het2)
3-(1H-1,2,3-triazol-5-yl)propanoic acid (TG-Het3)
2-(1H-tetrazol-5-yl)acetic acid (TG-Het4)
2-(5-nitro-4H-1,2,4-triazol-3-yl)acetic acid (TG-Het5)
   Amino-SynBase™ Controlled Pore Glass 3000/110 (LCAA) was functionalized with 2-(1H-tetrazol-5-yl)acetic acid
2-(5-nitro-4H-1,2,4-triazol-3-yl)acetic acid
   (Aminomethyl)polystyrene (AM-PS) was functionalized with
2-(1H-tetrazol-5-yl)acetic acid (AM-PS-Het4)
2-(5-nitro-4H-1,2,4-triazol-3-yl)acetic acid (AM-PS-Het5)
   PEGA was functionalized with
2-(1H-tetrazol-5-yl)acetic acid (PEGA-Het4)
   Aminomethyl ChemMatrix® (AM-CM) was functionalized with
2-(1H-tetrazol-5-yl)acetic acid (AM-CM-Het4)
   TentaGel® XV HMPA (TG-XV) Resin was functionalized with
2-(1H-tetrazol-5-yl)acetic acid (TG-XV-Het4)
   HypoGel® RAM Resin (HypoGel) was functionalized with
2-(1H-tetrazol-5-yl)acetic acid (HypoGel-Het4)
   Aminobutyl Polystyrene (AB-PS) was functionalized with
2-(1H-tetrazol-5-yl)acetic acid (AB-PS-Het4)

Imidazole functionalization of TentaGel™ S—NH₂ (TG-Het1):

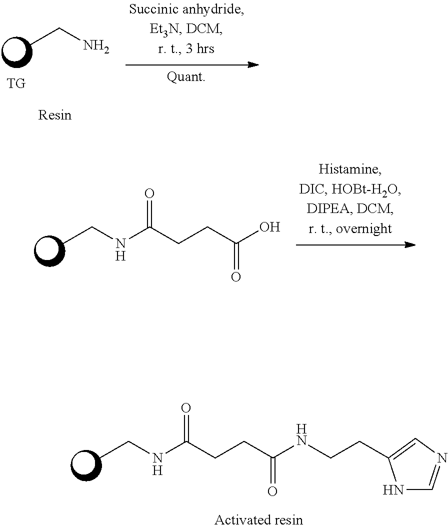

Succinic anhydride (100 mg, 1.0 mmol, 7.4 eq) was dissolved in CH₂Cl₂ (5 mL) and Et₃N (0.138 mL, 1.0 mmol, 7.4 eq) was added. The mixture was added to TentaGel™ S—NH₂ (300 mg, 0.45 mmol/g, 0.135 mmol, 1.0 eq) in a plastic tube and shaken at r.t. for 3 hrs. The resin was washed with CH₂Cl₂ and Et₂O. The beads were analysed by Kaiser test which gave negative results for amines meaning quantitative coupling yield.

A solution of DIC (0.11 mL, 0.68 mmol, 5.0), HOBt·H₂O (103 mg, 0.24 mmol, 1.8 eq) and DIPEA (0.35 mL, 0.72 mmol, 5.3 eq) in CH₂Cl₂ (4 mL) were added to the resin and shaken at r.t. for 30 minutes. Histamine (75 mg, 0.68 mmol, 5.0 eq) was then added to the mixture and tube was shaken overnight at r.t. The beads were then washed with MeOH, CH₂Cl₂ and Et₂O.

### Example 9—Loaded Resins

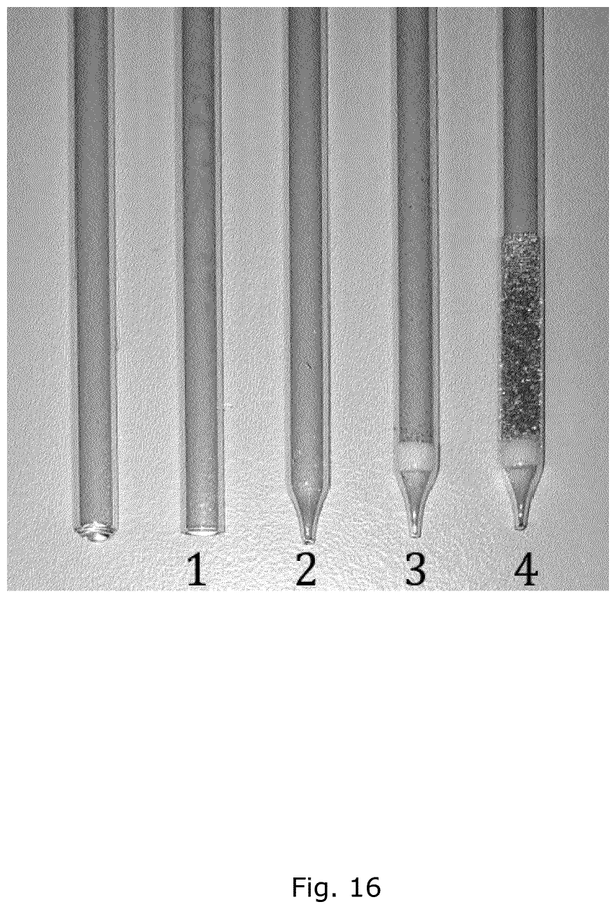

A modified NMR tube was created by the following procedure and the tube achieved after each of the numbered step in the procedure is depicted in FIG. 16. The bottom of an NMR tube (outer diameter: 5 mm) was cut off using a diamond tipped glass cutter (1). The glass was then melted under a torch and pulled with a tweezer (2). After cooling, a filter was added to the NMR tube (3).

The resin could then be added and used in the following experiments (4). The modified NMR tube was loaded with an activated resin (50 mg, 0.4 mmol/g, 0.2 mmol) and the resin washed with CH₂Cl₂ (2 mL). Then the activated resin was loaded by treatment with PCI (0.10 M) and N,N-diisopropylethylamine (DIPEA, 0.10 M) in DCM (2 mL) over 1 min and then washed again with DCM (2 ml).

³¹P NMR spectra for the loaded resins (gel phase after loading with PCI): TG-Het1, TG-Het2, TG-Het3, TG-Het4, and TG-Het5 were obtained.

Then, compounds matching the exposed part of a loaded resin (e.g. comprising the heterocyclic moiety and phosphitylating moiety), were synthesized according to the following reaction:

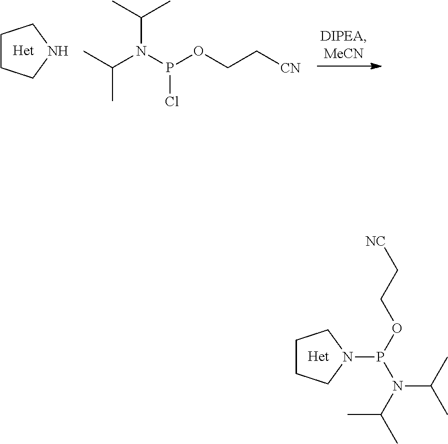

Heterocycles (0.10 M) were dissolved in MeCN (1.0 mL) and DIPEA (0.030 M) was added along with PCI (0.020 M).

³¹P NMR spectra of these compounds (solution phase) were obtained.

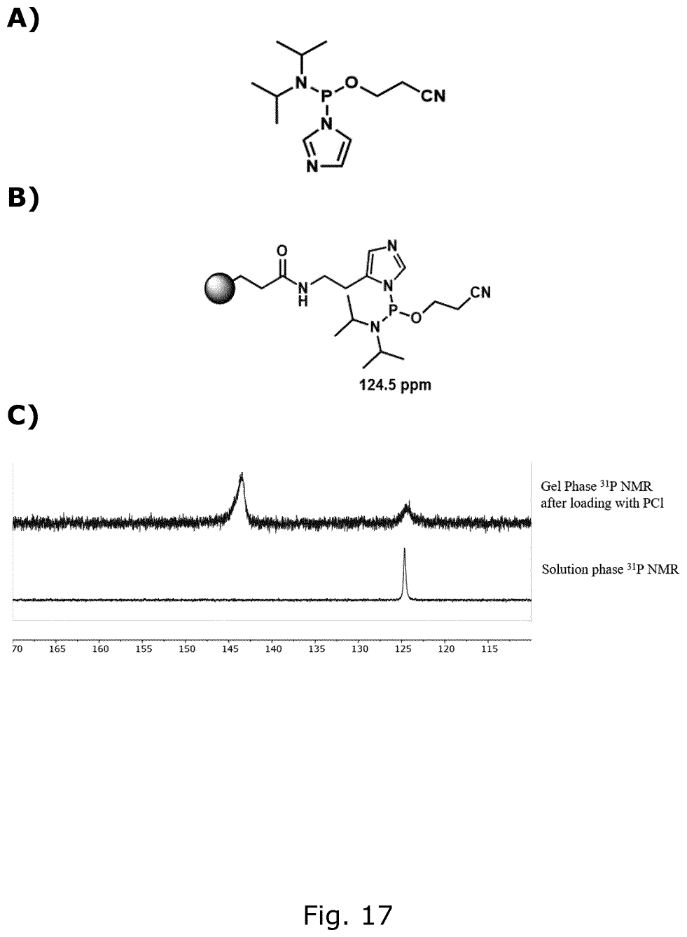

FIGS. 17 to 21 show, in inserts A) structures of molecules engineered to match the exposed part of a loaded resin, and in inserts B) structures of the corresponding loaded resins. Inserts C) show, two stacked ³¹P NMR spectra for characteristic peaks. The lower spectrum is of the structures depicted in A), whereas the upper spectrum is of the loaded resins in B). The characteristic NMR peaks (chemical shifts) for the P atom shifts or splits when connected to a specific resin. The resins were loaded with the PCI, chloro(diisopropylamino)(2-cyanoethoxy)phosphine.

FIG. 17 shows, that two peaks around 144 ppm and 125 ppm in the ³¹P NMR spectrum is characteristic for a loaded resin comprising imidazole.

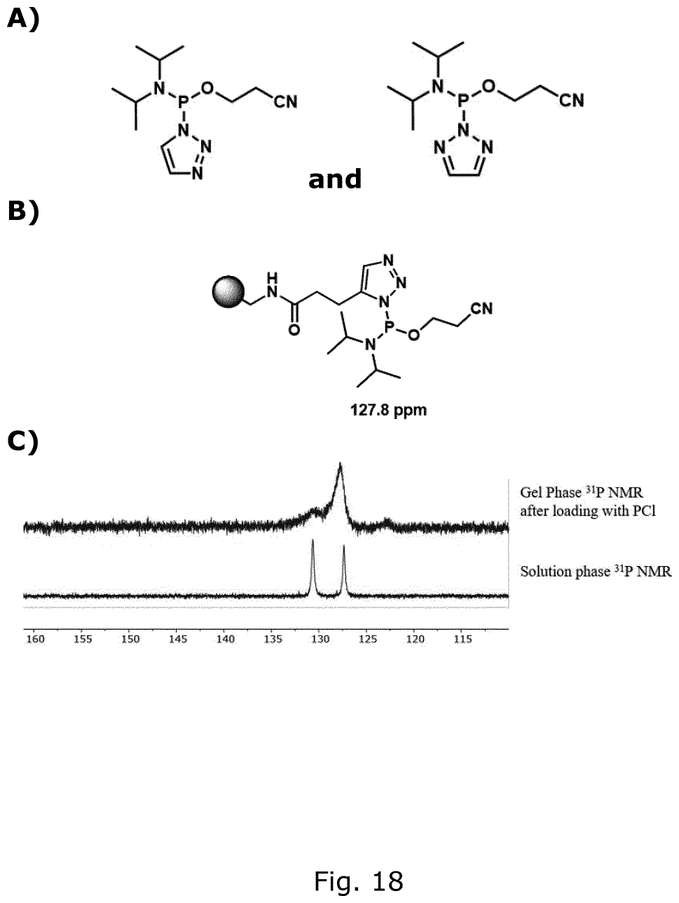

FIG. 18 shows, that a single broad peak around 128 ppm in the ³¹P NMR spectrum is characteristic for a loaded resin comprising 1,2,3-triazole.

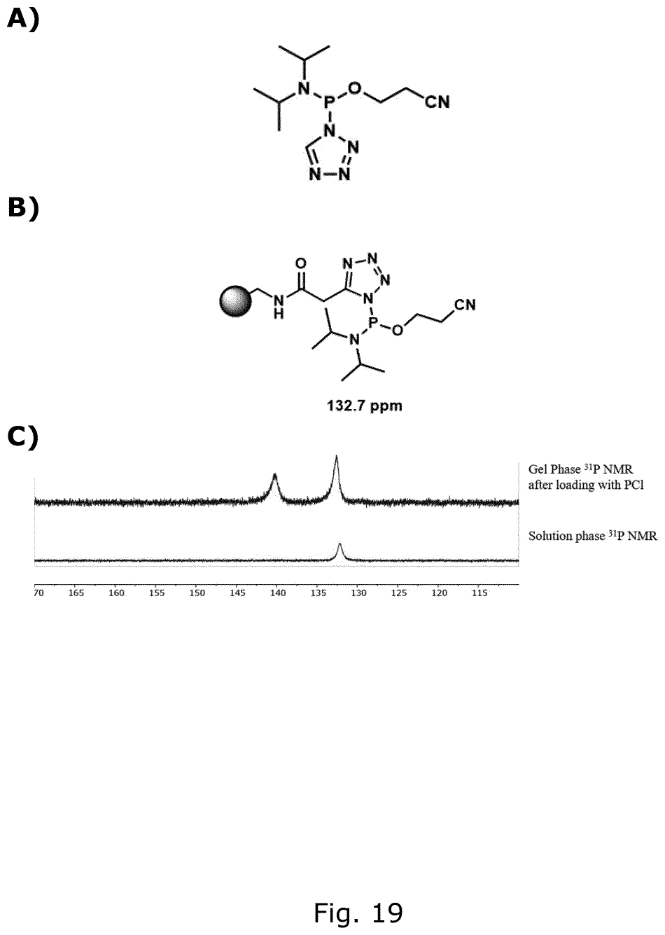

FIG. 19 shows, that two peak around 141 ppm and 133 ppm in the ³¹P NMR spectrum is characteristic for a loaded resin comprising tetrazole.

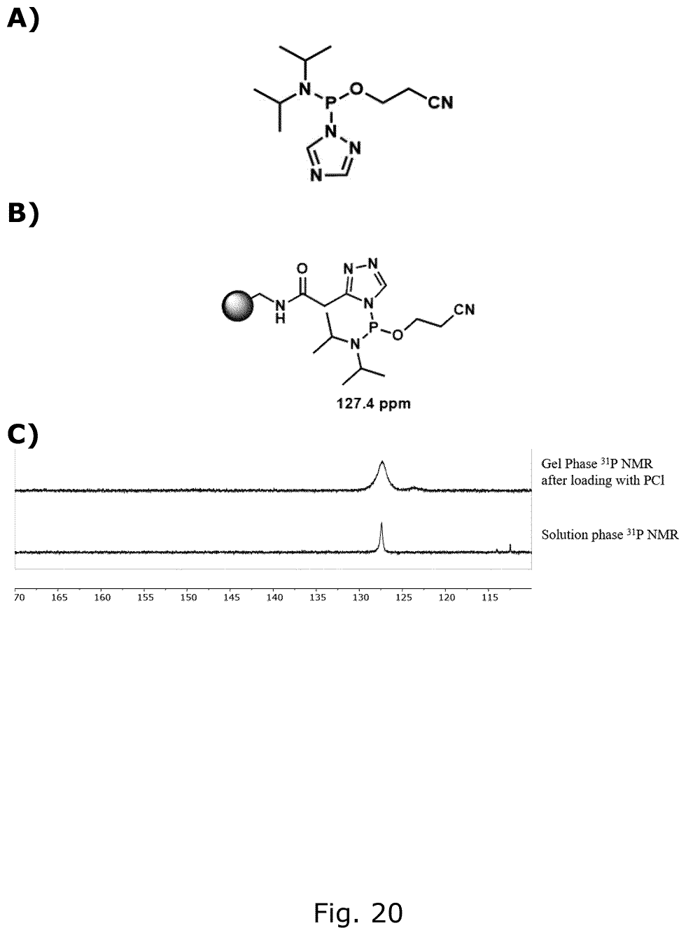

FIG. 20 shows, that a single broad peak around 127.5 ppm in the ³¹P NMR spectrum is characteristic for a loaded resin comprising 1,2,4-triazole.

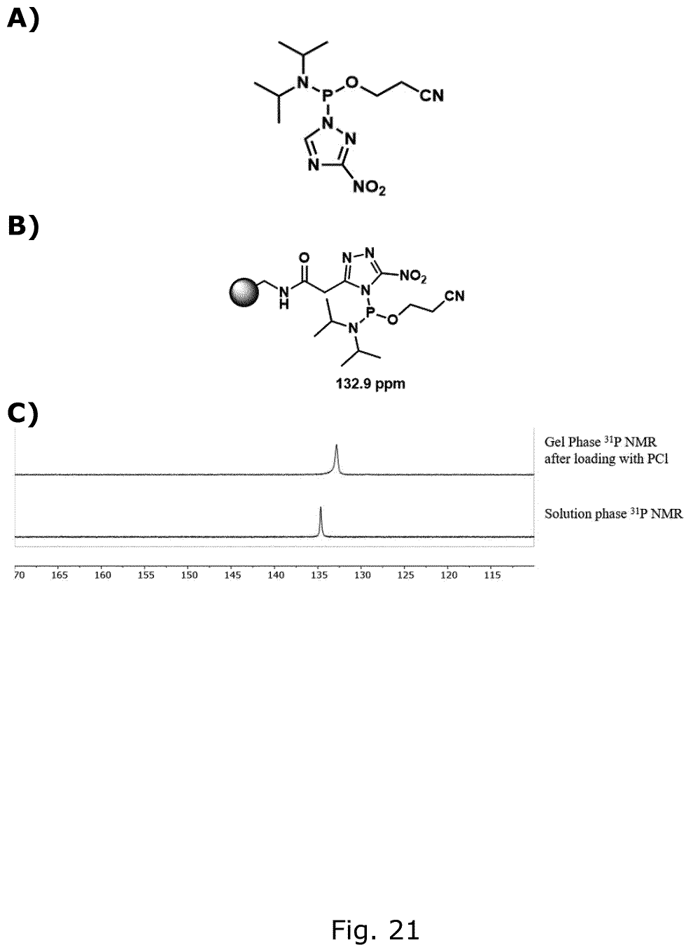

FIG. 21 shows, that a single broad peak around 133.5 ppm in the ³¹P NMR spectrum is characteristic for a loaded resin comprising 3-nitro-1,2,4-triazole.

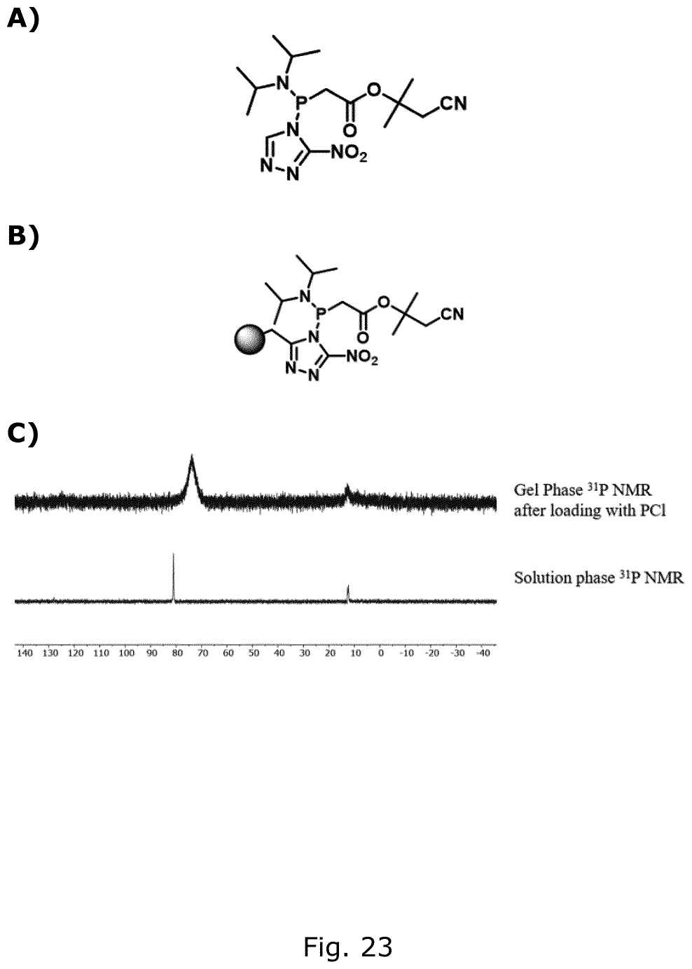

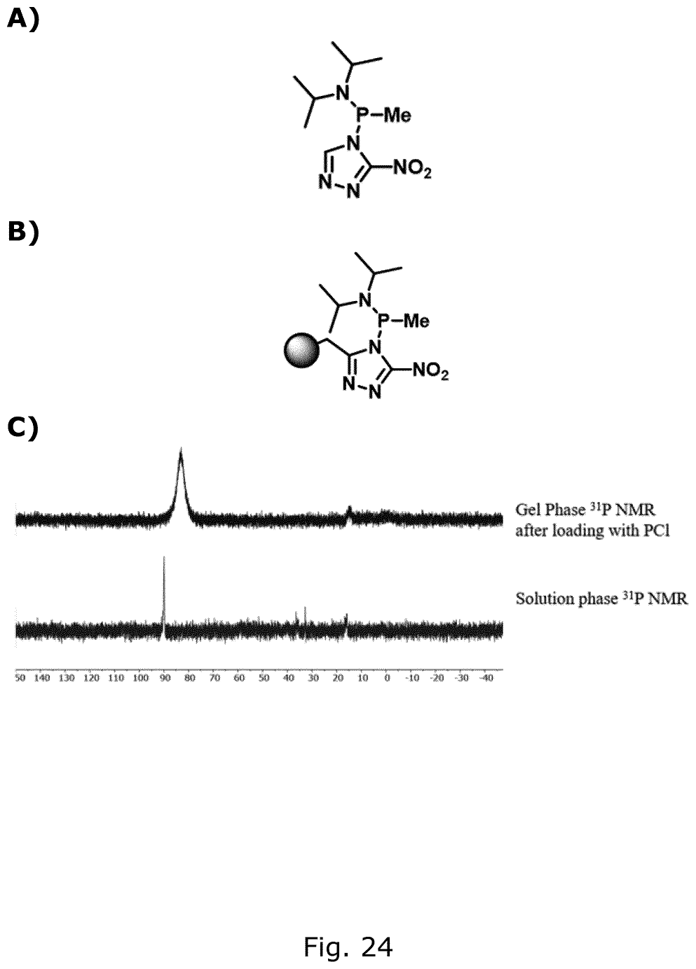

FIG. 23 to 24 show, in inserts A) structures of molecules engineered to match the exposed part of a loaded resin, and in inserts B) structures of the corresponding loaded resins. Inserts C) show, two stacked ³¹P NMR spectra for characteristic peaks. The lower spectrum is of the structures depicted in A), whereas the upper spectrum is of the loaded resins in B). The characteristic NMR peaks (chemical shifts) for the P atom shifts or splits when connected to a specific resin. The resin in FIG. 23 was loaded with the PCI, chloro(diisopropylamino)-(1,1-dimethyl-2-cyanoethoxycarbonylmethyl)phosphine, whereas the resin in FIG. 24 was loaded with the PCI, chloro(diisopropylamino)(methyl)phosphine. The observed changes in the ³¹P NMR chemical shifts between peaks relating to non-bound (in solution) and resin loaded PCI illustrate the characteristics for resins comprising 3-nitro-1,2,4-triazole and loaded with the respective PCI.

### Example 10—Residence Time with 9AJ as Base

The synthetic cycle used for screening of residence times is described in Table 9. The eluate was collected for 6 residence times and concentrated under reduced pressure. The crude mixtures were analysed 1H and 19F NMR (if possible) spectroscopy. Remaining conditions was as in example 4, unless otherwise stated.

TABLE 9
Summary of synthetic cycle

| Cycle | | Injection | Residence time | |
|---|---|---|---|---|
| step | Reagent(s) | volume | (flow rate) | Time |
| Loading | 0.1M PCl, 0.1M | 2.4 mL | 1 min | 10 mins |
| | DIPEA in CH₂Cl₂ | | (0.89 mL/min) | |
| Wash | CH₂Cl₂ | | 1 min | 5 mins |
| | | | (0.89 mL/min) | |
| Transfer | 0.104M Alcohol | 0.96 mL | Substrate | 6 residence |
| | (0.10 mmol), | | dependent | times |
| | 0.050M 9AJ in | | | |
| | CH₂Cl₂ | | | |

The yields were calculated by 1H NMR or 19F NMR (if possible) integration of non-overlapping signals between starting material and product. Results are given in Table 10.

TABLE 10
Summary of results from residence time study given as distributions
between phosphoramidite product and starting material.
Values ≥ 98% have been marked light green.

| | Residence time | | | | | | ³¹P NMR | ³¹P NMR |
|---|---|---|---|---|---|---|---|---|
| Alcohol | 1 min | 2 mins | 3 mins | 4 mins | 5 mins | 6 mins | purity* | purity** |
| Bz-dA | 78% | 95% | 99% | 100% | | | 81.0% | 96.9% |
| dT | 76% | 81% | 88% | 98% | 100% | | 88.9% | 94.9% |
| iBu-dG | 100% | | | | | | 72.1% | 93.5% |
| dmf-dG | 69% | 90% | 96% | 100% | | | 72.6% | 81.2% |
| Bz-dc | 72% | 88% | 92% | 97% | 97% | 98% | 93.3% | 97.6% |
| 1 | 90% | 99% | 100% | | | | 93.3% | 98.0% |
| 2 | 74% | 90% | 94% | >99% | | | 90.2% | 97.7% |
| 3 | 71% | 88% | 99% | 100% | | | 92.6% | 98.8% |
| 4 | 72% | 86% | 92% | 95% | 98% | 99% | 93.9% | 98.5% |
| 5 | 59% | 90% | 91% | 95% | 98% | | 94.9% | 98.9% |
| 6 | 100% | | | | | | 91.6% | 97.5% |
| 7 | 100% | | | | | | 92.3% | 96.0% |
| 8 | 100% | | | | | | 91.1% | 96.3% |
| 9 | 100% | | | | | | 91.1% | 94.5% |

*With lowest residence time where phosphoramidite/starting material distribution ≥ 98%
**Not counting the hydrolysis sideproduct at 13.8 ppm

The alcohols of table 10 are identified by structure in table 11 below. The PCI was chloro(diisopropylamino)(2-cyanoethoxy)phosphine as in example 4.

TABLE 11
alcohol structures

| Alcohol in | |
|---|---|
| table 10 | Structure |
| Bz-dA | 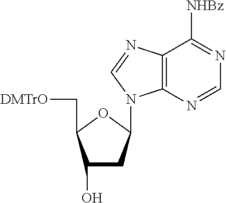 |
| dT | 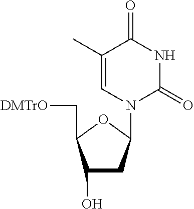 |
| iBu-dG | 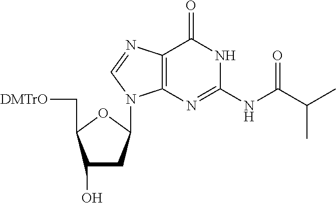 |
| Dmf-dG | 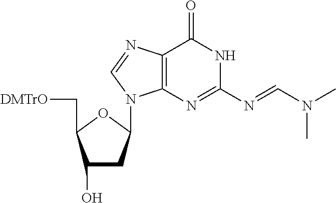 |
| Bz-dc | 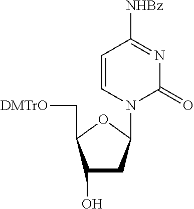 |
| 1 | 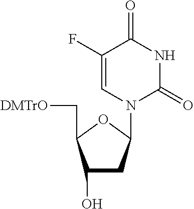 |
| 2 | 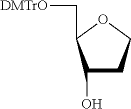 |
| 3 | 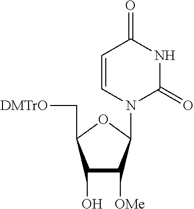 |
| 4 | 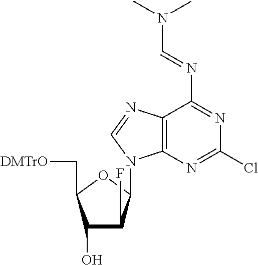 |
| 5 | 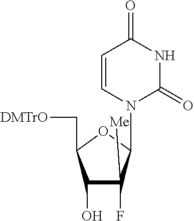 |
| 6 | 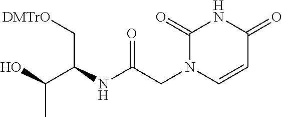 |
| 7 | 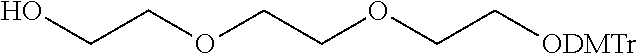 |
| 8 | 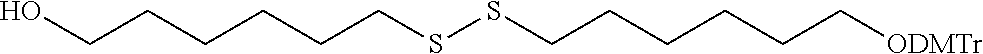 |
| 9 | 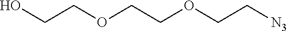 [SMILES: [N-]=[N+]=NCCOCCOCCO] |

### REFERENCES

J. Nielsen, M. Taagaard, J. E. Marugg, J. H. van Boom, O. Dahl, Application of 2-cyanoethyl N,N,N′,N′-tetraisopropylphosphorodiamidite for in situ preparation of deoxyribonucleoside phosphoramidites and their use in polymer-supported synthesis of oligodeoxyribonucleotides, Nucleic Acids Research, 1986, 14, 7391-7403.
C. G. Thomson, D. Le Grand, M. Dowling, C. E. Brocklehurst, C. Chinn, L. Elphick, M. Faller, M. Freeman, V. Furminger, C. Gasser, A. Hamadi, E. Hardaker, V. Head, J. C. Hill, D. I. Janus, D. Pearce, A. Poulad, E. Stanley, L. Sviridenko, Development of autotaxin inhibitors: A series of zinc binding triazoles, Bioorg. Med. Chem. Lett., 2018, 28, 2279-2284.
V. M. Chernyshev, A. V. Chernysheva, V. A. Taranushich, Optimization of the Synthesis of 5-Amino-1,2,4-triazol-3-ylacetic Acid and Bis(5-amino-1,2,4-triazol-3-yl)methane, Russ. J. Appl. Chem., 2009, 82, 276-281.
V. Thottempudi, J. M. Shreeve, Synthesis and Promising Properties of a New Family of High-Density Energetic Salts of 5-Nitro-3-trinitromethyl-1H-1,2,4-triazole and 5,5′-Bis(trinitromethyl)-3,3′-azo-1H-1,2,4-triazole, J. Am. Chem. Soc., 2011, 133, 19982-19992.
Molbiotools DNA calculator 2020: www.molbiotools.com/dnacalculator.html
N. Iwamoto, et al., Angewandte Chemie International Edition Vol 48, issue 3 (2009): 496-499.

1. A process for providing phosphoramidites, which process comprises the steps of:
a) providing an activated resin of a resin, comprising one or more of the heterocyclic moieties selected from the group consisting of imidazole, pyrazole, 1,2,3-triazole, 1,2,4-triazole, tetrazole, 2-nitrophenyltetrazole, 4-nitrophenyltetrazole, 2,4-dinitrophenyltetrazole, 3-nitro-1,2,4-triazole, 4,5-dicyanoimidazole, and 4-nitroimidazole,
b) obtaining a loaded resin by contacting the activated resin with a first solution comprising a first reactant according to Formula (1) 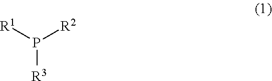
wherein, R¹ and R² are independently selected from the group consisting of di(C₁-C₆)alkylamino, pyrrolidino, morpholino, bromo, iodo, and chloro; R³ is selected from the group consisting of (C₁-C₆)alkyl, (C₁-C₆)alkoxy, 2-cyanoethoxy, benzoylthioethylthio, and 1,1-dimethyl-2-cyanoethoxycarbonylmethyl, or the first reactant is P-chloro-1-methyl-1-phenyl-2-oxa-3-phospha-tetrahydropyrrolizine,
c) contacting a second solution comprising a second reactant with the loaded resin of step b),
d) collecting the phosphoramidites,
wherein, the second reactant is selected from the group consisting of nucleosides, nucleoside analogs, synthetic nucleosides, disulfides, derivatives of glycols, azides, and ethylene glycol oligomers.

2. The process according to claim 1, wherein the second solution and the loaded resin in step c) are in contact with each other for 0.1 to 15 minutes.

3. The process according to claim 1, wherein the contacting of the second solution with the loaded resin in step c) is done by flowing the second solution through the loaded resin.

4. The process according to claim 1, wherein the phosphoramidites are at a purity which allows for direct utilization of the phosphoramidites in synthesis of oligonucleotides.

5. The process according to claim 1, wherein the first reactant is selected from the group consisting of
chloro(diisopropylamino)(2-cyanoethoxy)phosphine,
chloro(pyrrolidino)(benzoylthioethylthio)phosphine,
bis(diisopropylamino)(2-cyanoethoxy)phosphine,
chloro(diisopropylamino)(1,1-dimethyl-2-cyanoethoxycarbonylmethyl)phosphine,
bis(diisopropylamino)(1,1-dimethyl-2-cyanoethoxycarbonylmethyl)phosphine,
chloro(diisopropylamino)(methyl)phosphine,
bis(diisopropylamino)(methyl)phosphine, and
P-chloro-1-methyl-1-phenyl-2-oxa-3-phospha-tetrahydropyrrolizine.

6. The process according to claim 1, wherein the activated resin is selected from the group consisting of the following activated resins: 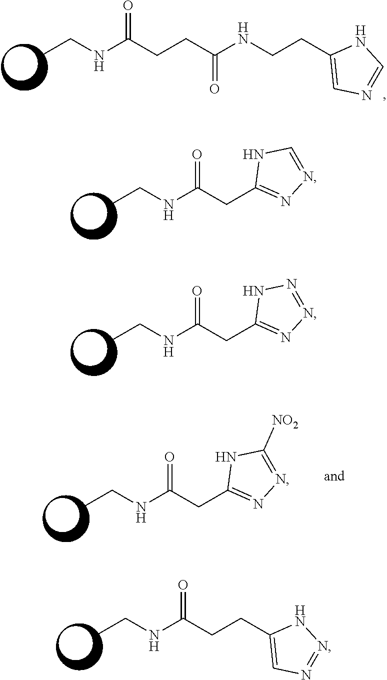
wherein ( [symbol] ) is the resin.

7. The process according to claim 1, wherein the second reactant is a compound according to a Formula selected from the group consisting of Formula (2), Formula (3), Formula (4), Formula (5), and Formula (6), 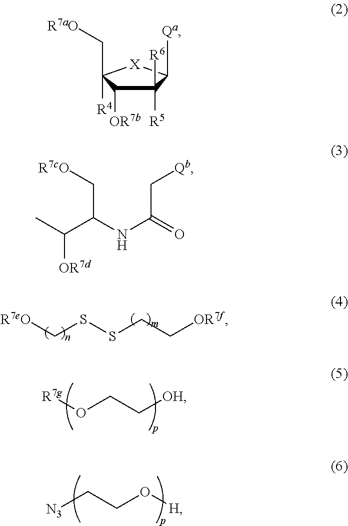
wherein, X is selected from the group consisting of —O—, —S—, —CH₂— and —NH—;
Q^{a} and Q^{b} are independently selected from the group consisting of hydrogen, and optionally substituted nitrogen heterocycle;
R⁴ is hydrogen or an optionally substituted (C₁-C₂)alkylidene bridge forming a ring together with R⁵; R⁵ is selected from the group consisting of —H, —OR⁸, —CH₃, —OCH₃, —OCH₂CH₃, —OCH₂CH₂OCH₃, —F, —Cl, —Br, and —I or forming a ring together with R⁴;
R⁶ is selected from the group consisting of —H, —OR⁹, —CH₃, —OCH₃, —F, —Cl, —Br, and —I; and
if R^{7a} is hydrogen then R^{7b} is an alcohol protecting group,
if R^{7b} is hydrogen then R^{7a} is an alcohol protecting group;
if R^{7c} is hydrogen then R^{7d} is an alcohol protecting group,
if R^{7d} is hydrogen then R^{7c} is an alcohol protecting group;
if R^{7e} is hydrogen then R^{7f} is an alcohol protecting group,
if R^{7f} is hydrogen then R^{7e} is an alcohol protecting group;
R^{7g}, R⁸ and R⁹ are alcohol protecting groups;
p and q are integers independently selected from the group consisting of 2, 3, 4, 5, 6, 7, 8 and 9;
n and m are integers independently selected from the group consisting of 1, 2, 3, 4, 5, 6, 7, and 8.

8. The process according to claim 7, wherein the nitrogen heterocycle is a radical of a compound selected from the group consisting of adenine, cytosine, guanine, thioguanine, thymine, uracil, xanthine, purine, pyrimidine, pyridazine, pyridine, pyrazine, triazine, pyrrole, pyrazole, imidazole, triazole, pyrrolopyrimidine, pyrazole[1,5-a]pyrimidine, azaindole, benzimidazole, phenoxazine, thiophenoxazine, indazole, indole, indoline, pyrrolopyrrole, quinoline, isoquinoline, theobromine, caffeine, uric acid, isoguanine, isocytosine, and hypoxanthine.

9. The process according to claim 7, wherein the nitrogen heterocycle comprises one or more optional substituents independently selected from the group consisting of hydrogen, (C₁-C₆)alkyl, (C₂-C₆)alkenyl, (C₂-C₆)alkynyl, (C₁-C₆)alkoxy, (C₁-C₆)alkylcarbonylamino, di(C₁-C₅)alkylaminomethaniminyl, phenoxyacetylamino, phenoxyacetylamino, p-isopropyl-phenoxyacetylamino, p-tert-butyl-phenoxyacetylamino, benzoylamino, 4,4′-dimethoxytrityloxy, t-butyldimethylsilyloxy, t-butylsilyloxy, bis(2-acetoxyethoxy)methoxy, 1,1-dioxo-thiomorpholin-4-thiocarbonyloxy, tri-iso-propylsilyloxymethoxy, trifluoromethyl, phenylcarbonyl, phenylcarbonylamino, isopropylcarbonyl, oxo, nitro, cyano, fluoro, chloro, bromo, and iodo.

10. The process according to claim 7, wherein the alcohol protecting groups are independently selected from the group consisting of 4,4′-dimethoxytrityl (DMTr), 4-methoxytrityl (MMTr), trityl (Tr), t-butyldimethylsilyl (TBDMS), t-butylsilyl (TBS), bis(2-acetoxyethoxy)methyl (ACE), 1,1-dioxo-thiomorpholin-4-thiocarbonyl, methoxymethyl (MOM), and tri-iso-propylsilyloxymethyl (TOM).

11. The process according to claim 1, wherein the loaded resin after step b) is free of the first solution.

12. An activated resin of a resin, comprising one or more of the heterocyclic moieties selected from the group consisting of imidazole, pyrazole, 1,2,3-triazole, 1,2,4-triazole, tetrazole, 2-nitrophenyltetrazole, 4-nitrophenyltetrazole, 2,4-dinitrophenyltetrazole, 3-nitro-1,2,4-triazole, 4,5-dicyanoimidazole, and 4-nitroimidazole.

13. A loaded resin comprising a resin connected to one or more of the heterocyclic moieties selected from the group consisting of imidazole, pyrazole, 1,2,3-triazole, 1,2,4-triazole, tetrazole, 2-nitrophenyltetrazole, 4-nitrophenyltetrazole, 2,4-dinitrophenyltetrazole, 3-nitro-1,2,4-triazole, 4,5-dicyanoimidazole, and 4-nitroimidazole, wherein each heterocyclic moiety is further connected to a phosphoramidite moiety.

14. A loaded resin obtained by a process comprising the steps of:
a) providing an activated resin of a resin, comprising one or more of the heterocyclic moieties selected from the group consisting of imidazole, pyrazole, 1,2,3-triazole, 1,2,4-triazole, tetrazole, 2-nitrophenyltetrazole, 4-nitrophenyltetrazole, 2,4-dinitrophenyltetrazole, 3-nitro-1,2,4-triazole, 4,5-dicyanoimidazole, and 4-nitroimidazole,
b) obtaining the loaded resin by contacting the activated resin with a first solution comprising a first reactant according to Formula (7) 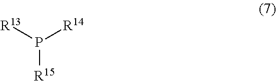
wherein, R¹³ and R¹⁴ are independently selected from the group consisting of di(C₁-C₆)alkylamino, pyrrolidino, morpholino, bromo, iodo, and chloro; R¹⁵ is selected from the group consisting of (C₁-C₆)alkyl, (C₁-C₆)alkoxy, 2-cyanoethoxy, benzoylthioethylthio, and 1,1-dimethyl-2-cyanoethoxycarbonylmethyl, or the first reactant is P-chloro-1-methyl-1-phenyl-2-oxa-3-phospha-tetrahydropyrrolizine.

15. The activated resin according to claim 12, wherein the activated resin is attached to a support selected from the group consisting of a column, tube, pipe, pipette, cylinder, funnel, porous glass, tubular container, needles, beads, pellets, powders, pearls, and grains.

16. The loaded resin according to claim 13, wherein the loaded resin is attached to a support selected from the group consisting of a column, tube, pipe, pipette, cylinder, funnel, porous glass, tubular container, needles, beads, pellets, powders, pearls, and grains.
